(19) United States
(12) Patent Application Publication
(10) Pub. No.: US 2007/0243537 A1
(43) Pub. Date: Oct. 18, 2007

(54) Human sample matching system

(76) Inventors: Edward F. Tuck, West Covina, CA (US); Martie G. Haselton, Culver City, CA (US); Mark A. Sturza, Encino, CA (US)

Correspondence Address: Giaccherini, Post Office Box 1146, Carmel Valley, CA, 93924, US

(21) Appl. No.: 11/405,001

(22) Filed: Apr. 14, 2006

(51) Int. Cl. C12Q 1/68; G06Q 50/00

(52) U.S. Cl. 435/6; 705/2

(57) ABSTRACT

Methods and apparatus for a collecting a human odor or DNA sample (S), analyzing the sample (S) and then determining a good romantic match is disclosed. In one embodiment, a customer purchases an AromaMatch™ Test Kit (14), which comprises a bottle of cleaning solution (20), a cotton ball (22) a sample patch (24), a sample bag (28) and a mailing envelope (30). The user (10) opens the kit (14), cleans a patch of skin somewhere on his or her body, and then applies the patch (24). After wearing the patch (24) for an appropriate time period, the patch (24) is removed, and placed in the sample bag (28). In an alternative embodiment, the user obtains a DNA sample using a cheek swab (42) or a spit cup (43). The user (10) writes his or her password on the sample bag (28) or some other container, and then mails it in the return envelope (30). A laboratory analyzes the sample patch or DNA sample (24), and determines a set of genetic attributes (G1) that are associated with the sample (S). The user (10) receives an analysis report by mail, or views the results of the analysis on a website (18), such as www.AromaMatch.com. In another embodiment, a customized perfume is manufactured that is based on biological agents that are selected to match the set of genetic attributes identified by the analysis. The perfume may be worn to stimulate sexual self-confidence or to enhance the attractiveness of another person.

### FIELD OF THE INVENTION

The present invention pertains to methods and apparatus for using a human sample to predict a good romantic match between a man and a woman. The human sample may comprise an odor or a DNA sample. More particularly, the invention is predicated upon the human female's enhanced sexual responsivity to men who possess a particular genetic profile that differs from her own, and the heightened attractiveness of either sex to such differing profiles. Specifically, a good romantic match between a man and a woman is determined by a strong correlation between attraction and the degree of dissimilarity of two partners' “Major Histocompatibility Complex (MHC)” alleles. In addition, a perfume may be formulated based on the analysis of genetic attributes. The perfume may either fortify the sexual self-confidence and broadcast the attributes of the person that wears the perfume, or may be used to enhance the attractiveness of another person.

### BACKGROUND OF THE INVENTION

Mammals have evolved efficient ways to find and select among potential mates. There has been a great deal of research on this subject in the twenty-three years since a landmark study found that mice choose their mates on the basis of their candidates' distinctive odors. Boyse E A, Beauchamp G K, Yamazaki K, et al., “Chemosensory Communication—A New Aspect of the Major Histocompatibility Complex and Other Genes in the Mouse.” Journal: Oncodevelopmental Biology and Medicine. Vol. 4 No. 1-2: pages 101-116, 1982. These odors are defined by the Major Histocompatibility Complex (MHC). The MHC is a cluster of genes that determines details of cellular surfaces and thus immune responses, and specifies certain peptides that appear in skin secretions and urine. These peptides are responsible for odors which, in essentially all cases, identify individuals who are not identical twins.

More recent work has shown that human female sexual responsivity to a male partner varies linearly and inversely with the degree to which alleles in the Major Histocompatibility Complex are shared. The correspondence is dramatic: about a nine-to-one difference in responsivity to men who share none of her MHC alleles, than to those who share sixty percent.

Men and women detect others' MHC genes through their body odors. There are a number of peptides that are derived from particular regions of the MHC. These peptides are detected as odors. They strongly affect a woman's responsivity to a particular partner, as discussed in the cited literature, and to both men's and women's mutual attractiveness.

This mate-selection process has a strong effect on the fitness of offspring. Choosing mates on the basis of MHC dissimilarity equips offspring with a broad immune system, increasing the offspring's fitness. It also selects against close relatives as mates, increasing the viability of fetuses and reducing birth defects. It also may reduce the rate of spontaneous abortion: there is some evidence that fetuses of couples which share significant numbers of MHC alleles are more likely to be rejected in utero. Zeh, Jeanne A. et al., “Outbred embryos rescue inbred half-siblings in mixed-paternity broods of live-bearing females,” Nature, Vol. 439, pp. 201-203 (12 Jan. 2006).

Other studies, including one cited above, have shown that women who are in long-term intimate relationships with men with similar MHCs are more likely to seek other partners during the fertile portion of their menstrual cycles. This practice obviously has a destabilizing effect on these relationships, which include marriages. Because humans' sense of smell is relatively poor, couples who are strangers must come into close personal contact before he or she can estimate their MHC-derived “fit” with a potential male partner and thus her long-term sexual responsivity to him. As humans have moved from villages to cities, various means have been created to bring men and women of marriageable age into close proximity under controlled conditions: examples range from the masked ball in Romeo and Juliet to modern on-line dating services. In modern human society, with much less class structure and much more freedom for men and women than in primitive, medieval or Victorian eras, this acquaintance process can pose considerable danger and risk of embarrassment to both women and men. The modern process of selecting a mate is very inefficient compared to these earlier societies, in which the number of potential partners available to each woman was comparatively small and where people lived in very close proximity. It would be of great benefit, not only to individual couples, but to society as a whole, if men and women could assess the sexual compatibility of prospective mates without coming into close contact. This is particularly important in view of the general failure of online dating services, which select matches without prior physical proximity or genetic matching, to produce acceptable results. See Gamerman, Ellen, “Mismatched.com,” in The Wall Street Journal Online, Apr. 1, 2006, page 1. Indeed, according to the referenced article, there is anecdotal evidence that pairings using online dating produce a larger-than-normal proportion of incompatible couples, leading to an increase in the general divorce rate in the United States. In contrast, pre-encounter MHC typing and comparison would, among other things, give women a wider range of prequalified candidates and would give men greater assurance that they and their prospective mates would have a stable and persistent relationship characterized by mutual physical attraction. It is generally conceded that mutual sexual attraction and responsivity are major contributors to pair bonding: they are the glue that holds long-term relationships together. People of all political and religious persuasions agree that stable pair-bonding, carrying the benefit of reduced strife and relationship discord, is in the best interest of society. Strife and relationship discord result in failed marriages and in infidelity. Society as a whole will thus benefit from easier and more accurate receptivity assessment. It is also important to note that there remain many cultures in which arranged marriages are the norm, and affianced couples do not meet before their wedding ceremony. Parents and matchmakers who are concerned with the success of their efforts could gain confidence from an MHC-based genetic matching process before a commitment is made.

Technology has advanced to the point that individual MHC-derived peptides, and thus odors, can be accurately detected artificially using gas chromatography and/or mass spectrometry (an “e-nose”). Willse, Alan et al., “Identification of Major Histocompatibility Complex-Regulated Body Odorants by Statistical Analysis of a Comparative Gas Chromatography/Mass Spectrometry Experiment,” Analytical Chemistry, Vol. 77, No. 8 (Apr. 15, 2005). This implies that a personal odor profile can be constructed for each individual, and that the degree of MHC sharing of two individuals can be derived by comparing those measurements, even if they are strangers and geographically distant from one another. MHC analysis can also be done on the basis of other material, such as cheek-cell scrapings, saliva samples and other means.

This process represents a considerable improvement to acquaintance-facilitation (“dating”) services based on the use of questionnaires and personality profiling. While these services help people find partners based on their subjective preferences and personality match, they say little about the likelihood of sexual attraction on first meeting, or the sexual responsivity of the partners in a long-term relationship. In contrast to these methods, MHC comparison is a completely objective process. Unlike current processes which rely on self-administered questionnaires, remote psychological assessments and other user-supplied personal data, MHC comparison cannot misrepresent its user.

Sexual dissatisfaction is the driving force behind Viagra® sales. No “female” Viagra® currently exists, despite the fact that women may be in greater need of it. A national probability sample study conducted by the AMA shows that 42% of women can be classified as having some sort of sexual disorder (Laumann et al., 1999). This rate is 75% higher than the rate of sexual dysfunction for men. The most common type of disorder for women is simple lack of interest in having sex. Garver-Apgar et al. (in press) also showed that women experienced more orgasms with partners who were MHC dissimilar. Orgasm disorder afflicts nearly 1 in 4 women. See Meston, Cindy et al., “Disorders of Orgasm in Women,” Journal of Sexual Medicine, Vol. 1 No. 1, pp. 66-68 (2004). See also Laumann, Edward et al., “Sexual Dysfunction in the United States, Prevalence and Predictors,” Journal of the American Medical Association, Vol. 281, No. 6, pp. 537-544 (1999).

The development of a system that could take advantage of this biological predilection of women to exhibit an enhanced attraction and sexual response to men who share her own genetic attributes would fulfill a long felt need in the dating and relationship industry, and would constitute a great benefit to members of society.

### SUMMARY OF THE INVENTION

The present invention provides a method and apparatus for taking a human sample, analyzing the sample and then determining a match with a member of the opposite sex. In one embodiment, a customer purchases an AromaMatch™ Test Kit, which comprises a bottle of cleaning solution, a sample patch, a sample bag and a mailing envelope. The user opens the kit, cleans a patch of skin somewhere on his or her body, and then applies the patch. After wearing the patch for an appropriate time period, the patch is removed, and placed in the sample bag. The user writes his or her password on the sample bag, and then mails it to a laboratory in the return envelope. The laboratory analyzes the sample patch, and determines a set of genetic attributes that are associated with the sample. The user receives an analysis report by mail, or views the results of the analysis on a website, such as www.AromaMatch.com.

In an alternative embodiment of the invention, a DNA sample may be obtained using a cheek swab or a spit cup. In another alternative embodiment, a kiosk or collector may be used to collect an aroma sample directly from the air, or from a cell sample collected locally.

In yet another embodiment, the customer may purchase a custom-formulated perfume, cologne, salve or other cosmetic or preparation that contains enhanced aromas that match his or her genetic attributes, or which complement her own genetic attributes; and when applied to herself or her partner, to enhance her sexual responsivity to her current partner, or to amplify the attractiveness of another person.

An appreciation of the other aims and objectives of the present invention, and a more complete and comprehensive understanding of this invention, may be obtained by studying the following description of preferred and alternative embodiments, and by referring to the accompanying drawings.

### A BRIEF DESCRIPTION OF THE DRAWINGS

FIG. 1 shows a woman purchasing an AromaMatch™ Test Kit at a retail store.

FIG. 2 shows the same woman purchasing an AromaMatch™ Test Kit on a website.

FIG. 3 depicts the woman opening the AromaMatch™ Test Kit to reveal its contents: a bottle of cleaning solution, a cotton ball, a sample patch, a sealable plastic bag and a mailing envelope.

FIG. 4 supplies a detailed view of the sample patch.

In FIG. 5, the woman cleans a patch of skin in preparation for applying the sample patch to her arm.

In FIG. 6, the woman applies the patch to her forearm.

FIG. 7 shows the woman wearing the patch all day.

FIG. 8 portrays the woman removing the sample patch from her arm later that evening.

In FIG. 9, the woman places the sample patch that she has worn for a day into the bag, and seals it.

In FIG. 10, the woman writes her password on the sealable bag.

In FIG. 11, the sample that has been sealed in the bag is placed in a mailing envelope.

FIG. 12 shows the woman mailing an envelope which contains the bag, which, in turn, contains the worn sample patch.

FIG. 13 shows a laboratory technician using an analyzer to determine the genetic attributes of the tissue sample that has been received from the woman depicted in FIG. 12.

In FIG. 14, the woman uses her computer to visit a website to obtain the results of the laboratory analysis.

In FIG. 15, the website reports the results of a matching process that has been performed using a library of candidates.

FIG. 16 exhibits an alternative embodiment, which collects a sample directly from the air surrounding a man.

FIG. 17A reveals yet another alternative embodiment, in which a tissue sample is obtained using a cheek swab.

FIG. 17B reveals an embodiment of the invention in which a saliva sample is obtained using a spit cup.

In FIG. 18, a woman whose tissue sample has already been analyzed receives a custom-formulated perfume which contains aromas that are correlated with her genetic attributes.

FIG. 19 shows a man using a MateFinder™ device which has been programmed with his genetic attributes, as determined in accordance with the present invention.

FIG. 20 shows a woman visiting a doctor's office or an independent laboratory to provide a tissue sample, which is then certified by the doctor or laboratory technician before it is submitted for analytic comparison.

FIG. 21 depicts a method of manufacturing a customized perfume.

FIG. 22 depicts a cell.

FIG. 23 illustrates a nucleus in a cell.

FIG. 24 portrays a chromosome inside the nucleus.

FIG. 25 exhibits a gene, which is a portion of a chromosome.

FIGS. 26 and 27 exhibit strands of DNA.

FIG. 28 furnishes a view of a chain of amino acids.

FIG. 29 reveals the growth of a polypeptide chain of amino acids.

FIG. 30 offers a view of the function of alleles.

FIG. 31 shows how a sample patch is cut into three disc-shaped pieces.

FIG. 32 illustrates the method of placing the three disc-shaped pieces into test tubes for processing and analysis.

FIG. 33 is a chart showing the relationship of alleles in the MHC Group on human Chromosome Number 6.

FIG. 34 is a schematic illustration of test results based on the analysis of one of the disc-shaped samples.

FIG. 35 describes a Human Leukocyte Antigen.

FIG. 36 describes Diploid Haplotypes.

FIG. 37 depicts MHC Allele Groups.

FIG. 38 describes a Similarity Metric.

### A DETAILED DESCRIPTION OF PREFERRED & ALTERNATIVE EMBODIMENTS

I. The AromaMatch™ System

The AromaMatch™ System provides methods and apparatus for determining a good romantic match based on a collected sample of human body odor or a DNA sample.

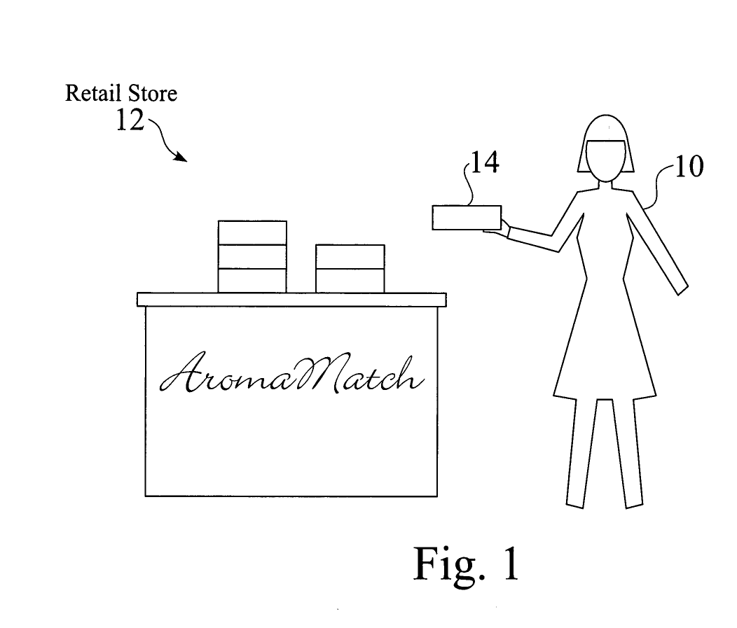

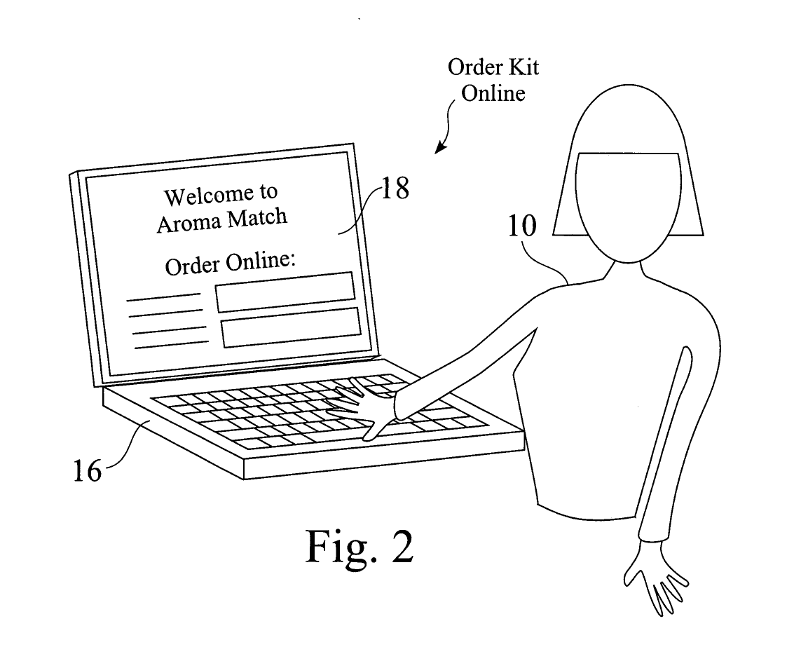

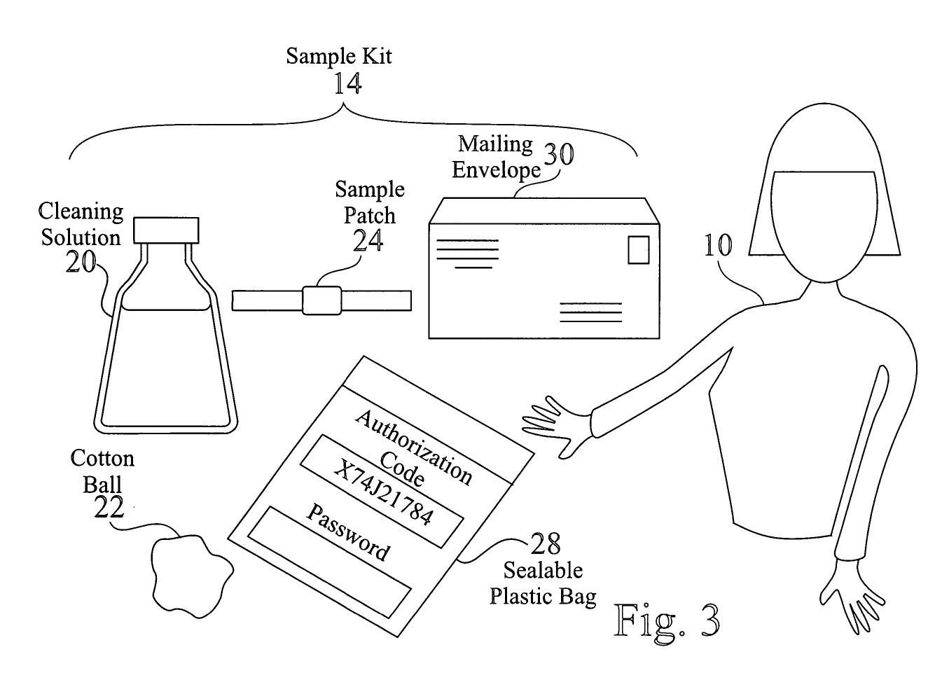

FIG. 1 offers a view of a woman 10 in a retail store 12, where she purchases an AromaMatch™ Test Kit 14. As an alternative, the woman 10 may purchase the same AromaMatch™ Test Kit 14 by visiting a website, www.AromaMatch.com, using her personal computer, as shown in FIG. 2. “AromaMatch” is a Trade & Service Mark owned by the Assignee of the Present Patent Application. The website “www.aromamatch.com” is also owned by the Assignee of the Present Patent Application. In this Specification and in the Claims that follow, the term “aroma” encompasses any scent, smell, odor or olfactory component that may or may not be actively or consciously detected, sensed or smelled by a person.

The woman opens the Test Kit 14 and removes the contents in FIG. 3. In one embodiment of the invention, the Test Kit 14 comprises:
skin cleaner 20;
a cleaning medium 22;
a sample patch 24;
a sealable enclosure 28; and
a mailing envelope 30.
   The skin cleaner 20 may comprise a liquid cleaning solution such as isopropyl alcohol, or any other, gel, solid, spray or substance that cleans and/or sterilizes a portion of the skin. The application of the skin cleaner 20 removes or neutralizes perfumes and other irrelevant smells.

The cleaning medium 22 is generally a small portion of material that is used to apply the skin cleaner 20 to the skin. In one embodiment, the cleaning medium 22 may be a cotton ball, wad, paper, piece of fabric or some other suitable application device.

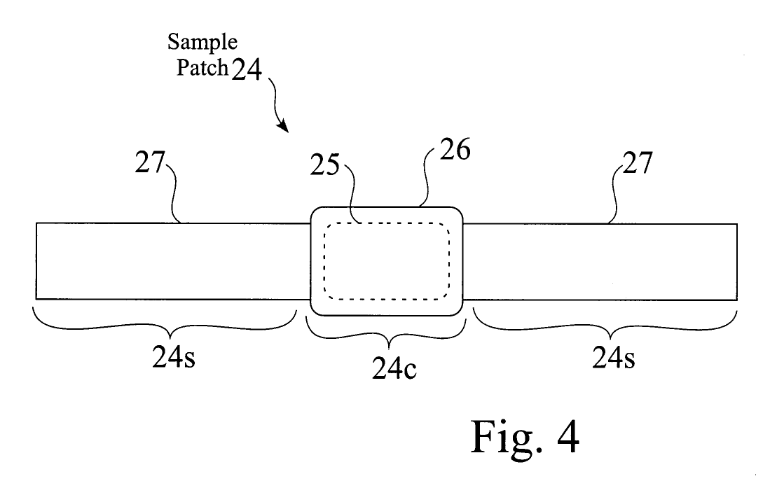

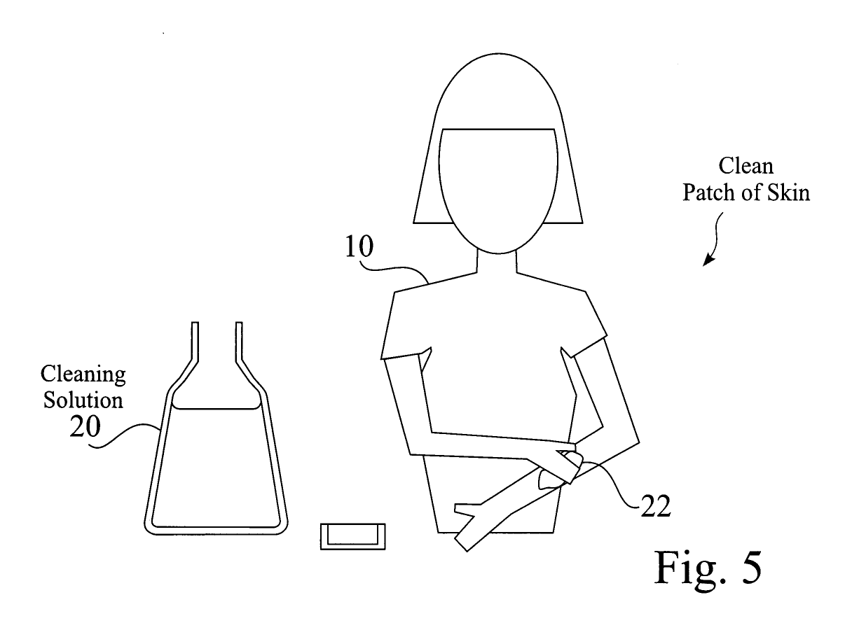

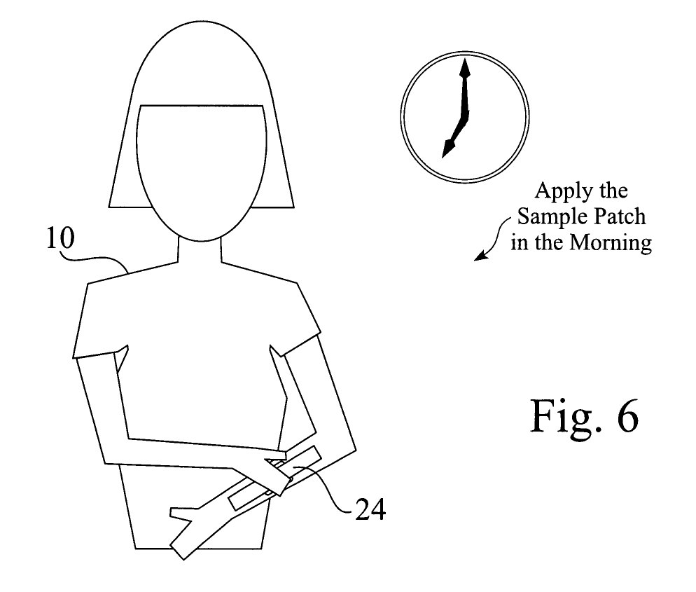

FIG. 4 furnishes an illustration of the sample patch 24, which comprises a small central area 24C with two outwardly extending strips 24S. The central area 24C is coated with a portion of plaster 25 which, in turn, has been coated with an antibiotic 26 or some other suitable agent that prevents bacterial growth which might modify the aroma. The strips 24S on either side of the plaster 25 are coated with an adhesive 27 that is suitable for adhering to the skin for a short period of time.

In one embodiment, the patch 24 resembles a conventional “Band-Aid® Brand” Adhesive Bandage, such as that manufactured and sold by Johnson & Johnson of New Brunswick, N.J. The patch 24 may be fabricated from plastic, cloth, paper or any other material that will maintain the plaster 25 in generally continuous contact with the skin. The plaster 25 is generally any material that will absorb and then hold an aroma which has been secreted by the skin. The plaster 25 may be composed of any substance that collects and stores an aroma. In one embodiment of the invention, the plaster 25 is manufactured from any material that may be used as an odor-absorbing poultice.

The plaster 25 is designed so that it will collect enough aromas to provide a sample which may be reliably analyzed. The aromas captured by the plaster 25 must be able to survive for a duration of time that is required for the patch 24 to be mailed to a laboratory.

After the Test Kit 14 is opened, the woman 10 cleans a patch of skin on her arm in preparation for applying the sample patch 24, as shown in FIG. 5. In FIG. 6, patch 24 has been attached to her forearm. The patch 24 may be worn on any portion of the body which allows direct and intimate contact with the skin. In some instances, selecting a patch of skin on or near the armpits may be produce optimal results.

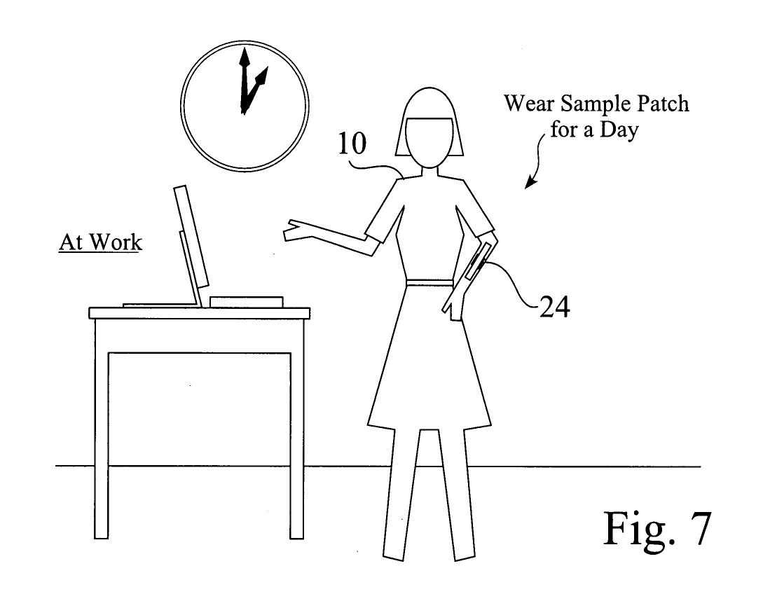

The woman wears the patch 24 all day, as shown in FIG. 7. The time that is required for the patch 24 to remain in place varies with the effectiveness of the plaster 25 and the sensitivity of the equipment used to analyze the patch 24. In one embodiment of the invention, the user is instructed to leave the patch 24 in place on the skin for at least eight hours. In some instances, the time that is required to wear to patch to obtain a good sample may take longer. One alternative method that may be used to collect a sample is simply wearing a shirt or some other article of clothing for an extended time, and then analyzing this worn article of clothing.

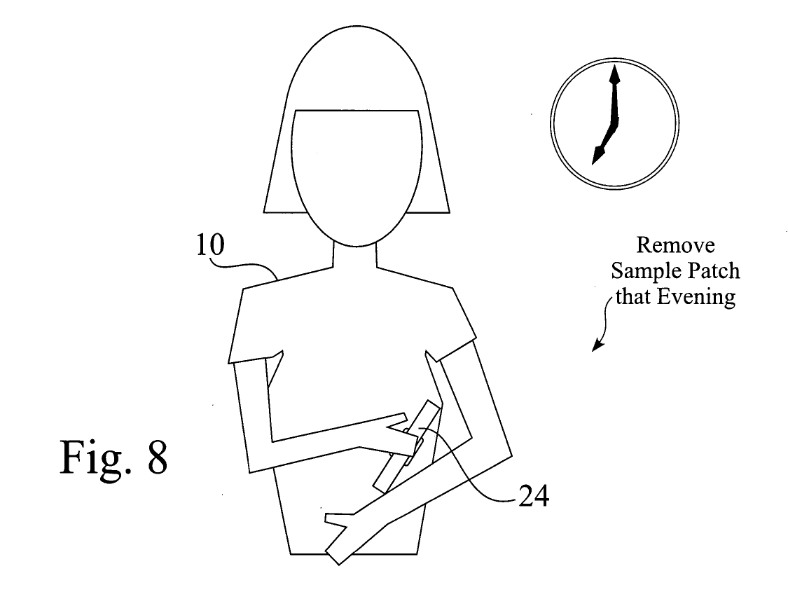

After wearing the patch 24 all day, the woman 10 removes the patch 24 later that evening, as shown in FIG. 8. After the patch 24 is removed, she then immediately places the patch 24 in the enclosure 28, as illustrated in FIG. 9. The enclosure is sealed 28 to prevent any degradation of the aromas stored in the plaster 25.

She then writes her username, password, code or some other identifying information on the bag 28, as shown in FIG. 10. This enclosure 28 is large enough to hold the sample patch 24, may be easily sealed against the intrusion of outside air by the user, and is generally an impermeable container or barrier that preserves the aromas imparted to the plaster 25 on the patch 24. In one embodiment of the invention, the enclosure 28 is a plastic bag with a compression seal, which is commonly known as a “zip-lock” or “slide-lock” closure. In one implementation, the bag 28 bears a pre-printed authorization code.

The patch 24 which stores the sample S which has been sealed in the bag 28 is then placed in the mailing envelope, as shown in FIG. 11.

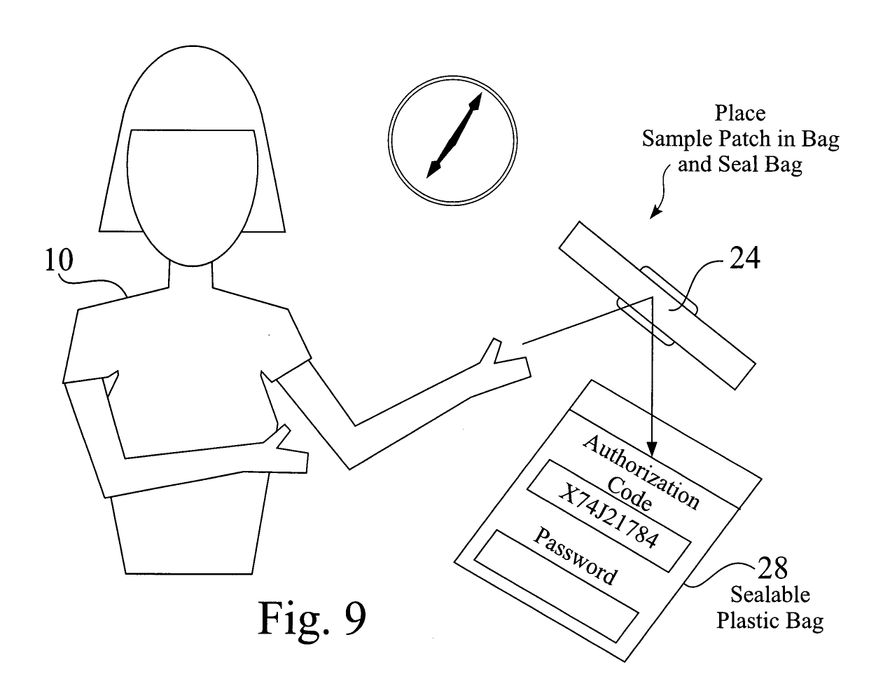

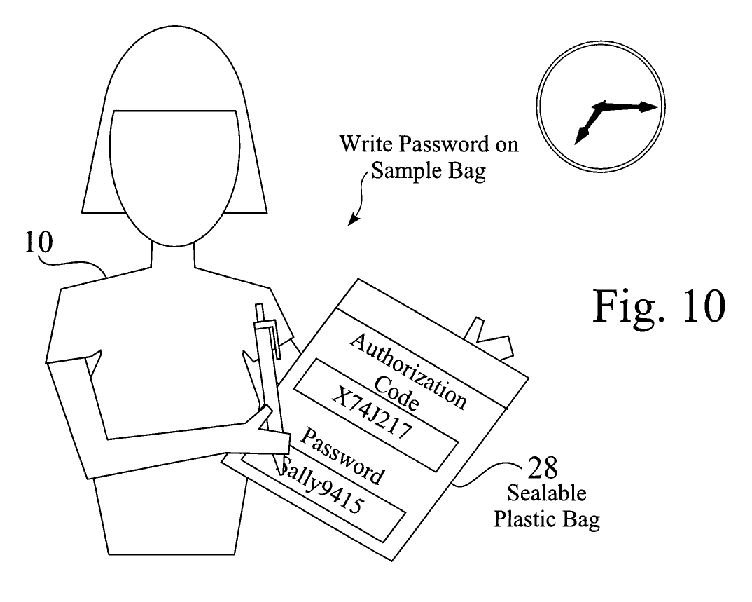

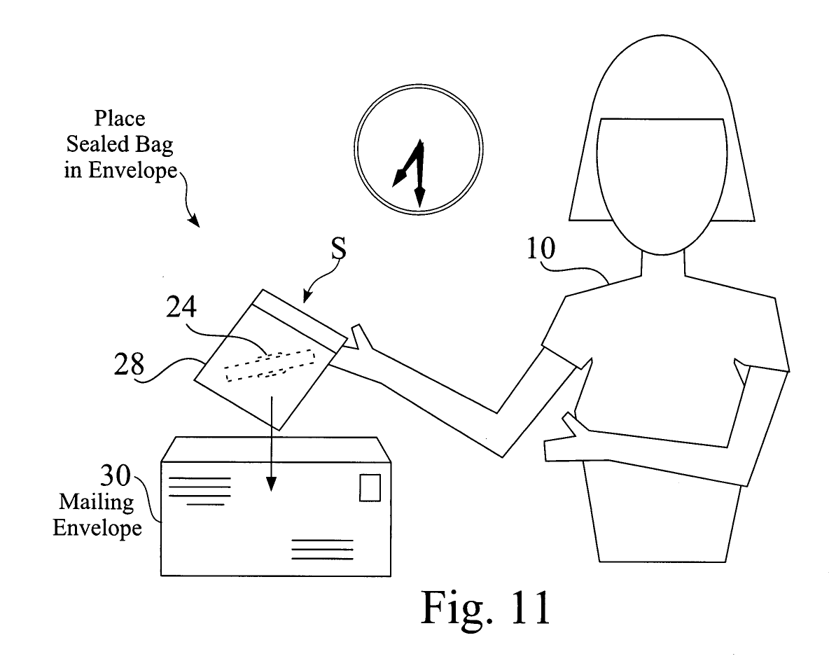

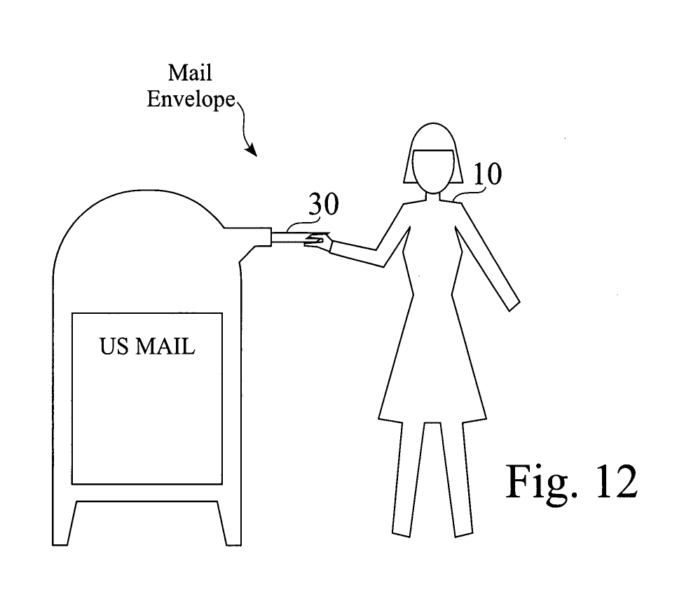

FIG. 12 portrays the customer posting the pre-addressed mailing envelope 30 which contains the worn patch 24 in the bag 28. This envelope 30 will convey the patch 24 to a laboratory where the plaster 25 will be analyzed. As an alternative, the patch 24 may be shipped to a laboratory using a courier. The patch 24 may also be delivered to a local lab, doctor's office or pharmacy for analysis. In a more advanced embodiment of the invention, the user may analyze the patch 24 using a home analysis kit.

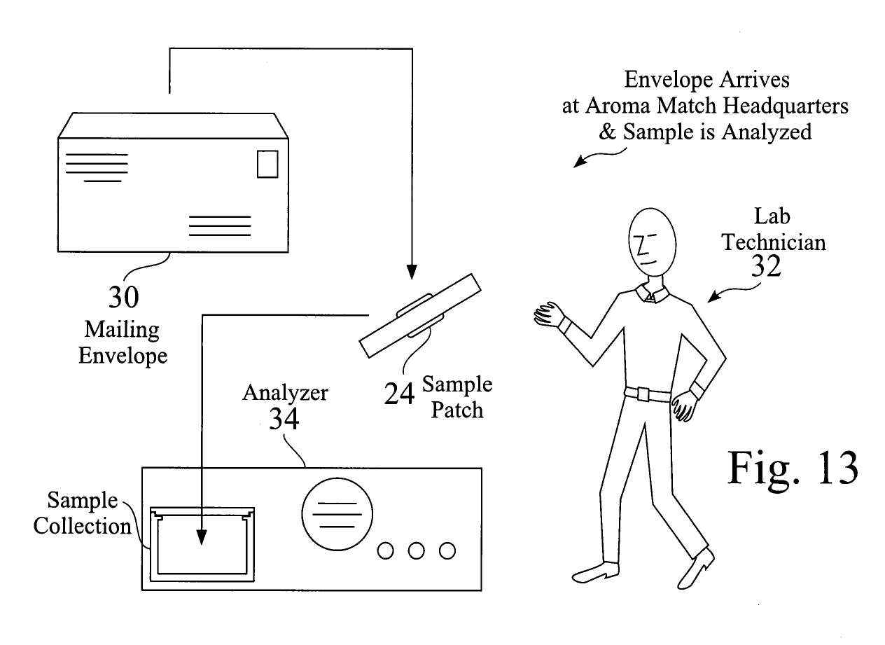

FIG. 13 shows a laboratory technician 32 using an analyzer 34 to determine the genetic attributes of the tissue sample that has been received from the customer 10. In one embodiment, a probe from an analyzer 34 may be inserted into the bag 28, which will convey the aromas to a chamber where a chemical analysis is conducted.

Several devices and systems for analyzing a sample are currently available which may be used to implement the present invention. One device called an “Electronic Nose” has been described by The Lewis Group of The California Institute of Technology, and is based on readily fabricated, chemically sensitive conducting polymer films. According to information presented on their website:
“An array of sensors that individually respond to vapors can produce a distinguishable response pattern for each separate type of analyte or mixture. Pattern recognition algorithms and or neural network hardware are used on the output signals arising from the electronic nose to classify, identify, and where necessary quantify, the vapor or odors of concern. This response is much like the way the mammalian olfactory sense produces diagnostic patterns and then transmits them to the brain for processing and analysis.
This approach does not require development of highly specific recognition chemistries, one for each of the many possible analytes of interest. Instead this approach requires a broadly responsive array of sensors that is trainable to the target signature of interest and then can recognize this signature and deliver it to the sensing electronics in a robust fashion for subsequent processing by pattern recognition algorithms. The Caltech electronic nose functions at atmospheric pressure, functions in a variety of ambients, exhibits near-real time detection, and has already been demonstrated to track vapors in air.
The underlying principle of the Caltech electronic nose is extraordinarily simple. When a polymer film is exposed to a gaseous vapor, some of the vapor partitions into the film and causes the film to swell. In the electronic nose, this swelling is probed electrically because the sensor films each consist of a composite that contains regions of a conductor that have been dispersed into the swellable organic insulator. The vapor-induced film swelling produces an increase in the electrical resistance of the film because the swelling decreases the number of connected pathways of the conducting component of the composite material. The detector films can be formed from conducting polymer composites, in which the electronically conductive phase is a conducting organic polymer and the insulating phase is an organic polymer, or from polymer-conductor composites in which the conductive phase is an inorganic conductor such as carbon black, Au, Ag, etc and the insulating phase is a swellable organic material. The electrical resistance of the device is then read using simple, low power electronics.
Any individual sensor film responds to a variety of vapors, because numerous chemicals will partition into the polymer and cause it to swell to varying degrees. However, an array of sensors, containing different polymers, yields a distinct fingerprint for each odor because the swelling properties over the entire array are different for different vapors. The pattern of resistance changes on the array is diagnostic of the vapor, while the amplitude of the patterns indicates the concentration of the vapor.”
   See: The Lewis Group, California Institute of Technology, Pasadena, Calif. Webpage: http://nsl.caltech.edu/resnose.html

A second device that may be used to implement the present invention is called the “Cyranose,” and is described by Rodney M. Goodman, in his article entitled “The Electronic Nose.” According to Goodman:
“Cyranose
The technology uses sensors mixed with carbon black to make them conductive. The polymers swell with an odorant and their resistance changes. An array of different polymers swell to different degrees giving a signature of the odorant. This technology has been commercialized by Cyrano Sciences (http://cyranosciences.com) and a handheld electronic nose has been launched as a product.”
   See: http://www.rodgoodman.ws/electronic_nose.html

A third device that may be used to implement the present invention is described by Smiths Detection of Danbury, Conn., which produces and sells devices for identifying materials. See: http://www.sensir.com/Smiths/Home.htm.

In FIG. 14, the customer 10 uses her computer 16 to visit a website 18 to obtain the results of the laboratory analysis. In one embodiment, the analysis includes a listing of MHC alleles, MHC-determined peptides, MHC-odors or some other MHC-dependent profile. In an alternative embodiment, the results may be dispatched to the customer by regular mail or by e-mail.

In an alternative embodiment of the invention, the customer pays for the Test Kit 14 and the analysis when he or she obtains the results of the analysis.

In FIG. 15, the website 18 reports the results of a matching process that has been performed using a library of candidates.

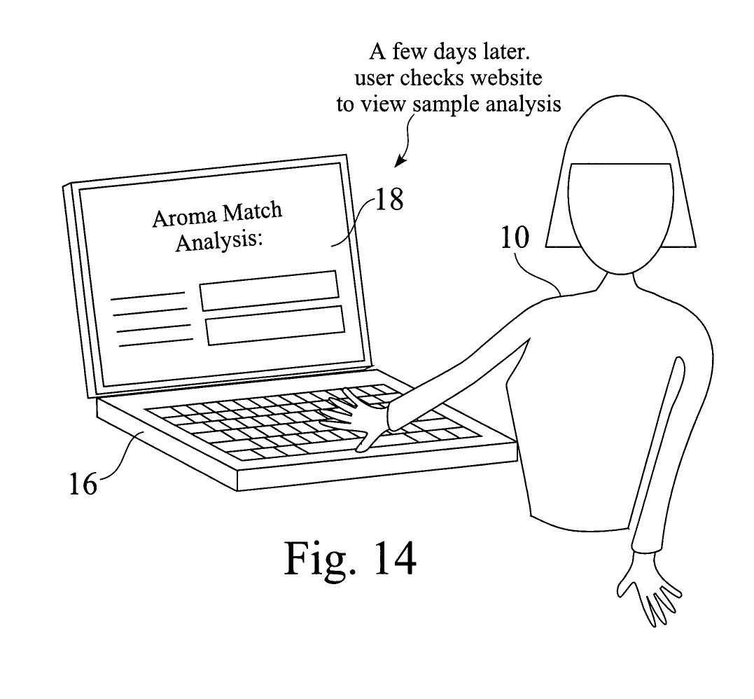

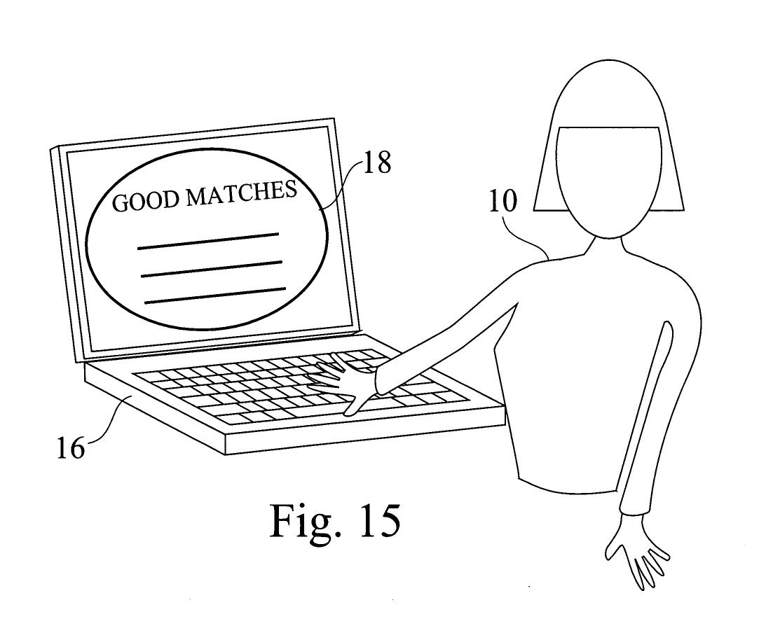

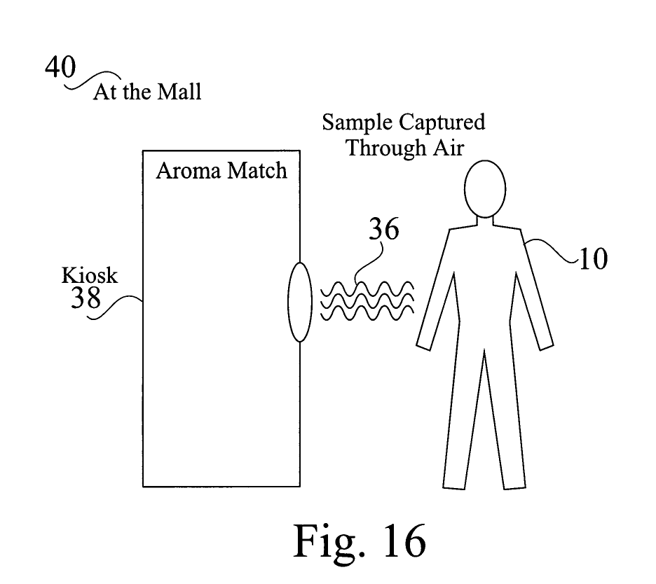

FIG. 16 exhibits an alternative embodiment, which collects a sample directly from the air 36 surrounding a customer 10 standing near a kiosk 38 that has been installed in a shopping mall 40. In yet another embodiment, a sample collecting tube may briefly be placed under a portion of a customer's clothing to obtain an air sample.

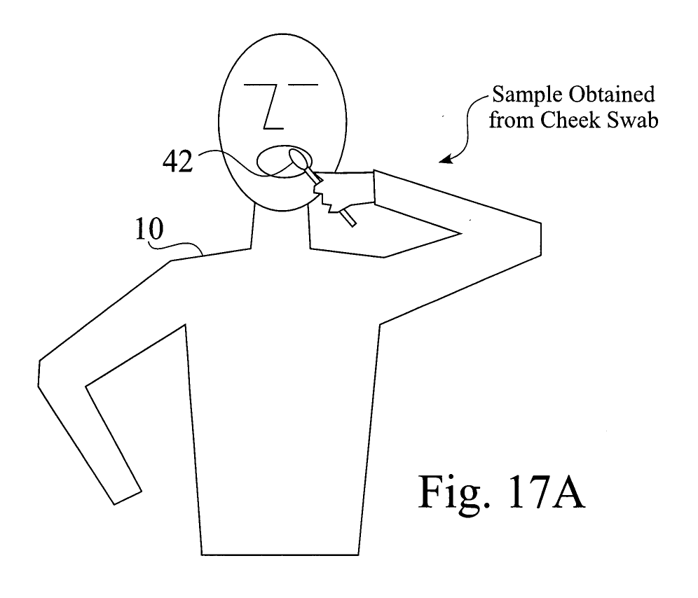

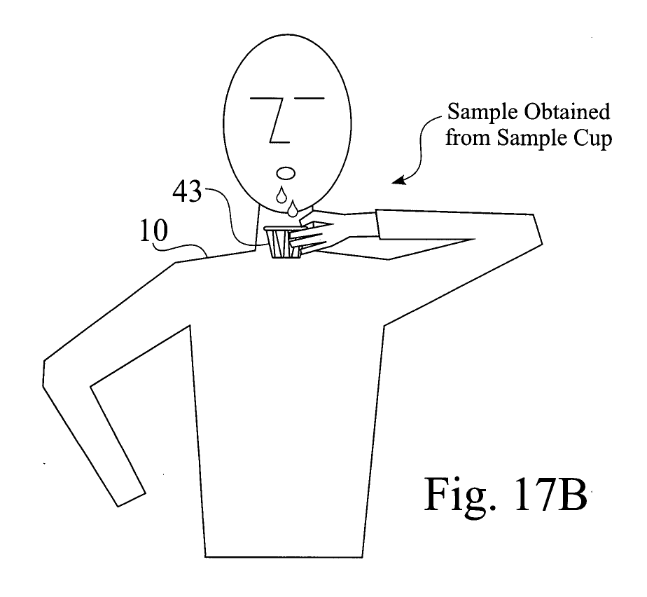

FIG. 17A reveals yet another alternative embodiment, in which a tissue sample 42 is obtained using a cheek swab. In other embodiments, a tissue sample may be obtained from any suitable bodily material or fluid, including, but not limited to, blood, saliva, exhaled breath, fingerprint, urine, hair, nail, or skin. One device that may be used to implement this portion of the present invention is produced and sold by DNA Genotek of Ottowa, Ontario, Canada, which produces and sells the Oragene™ DNA Self-Collection Kit, for collecting and preserving large amounts of DNA from saliva. See: http://www.dnagenotek.com/

In another embodiment of the invention, DNA may be collected from a customer by obtaining a sample of saliva in a container such as a spit cup 43, as shown in FIG. 17B.

In an alternative embodiment of the invention, an automatic machine or device which accepts a DNA sample may be used to obtain an analysis without the intervention of a technician or clerk.

In FIG. 18, a customer whose tissue sample has already been analyzed receives a custom-formulated perfume 44, “MyAroma™” or “MyCologne™,” which contains olfactory reagents that are correlated with his or her genetic attributes, and specifically, which are correlated with his or her MHC-derived peptide profile.

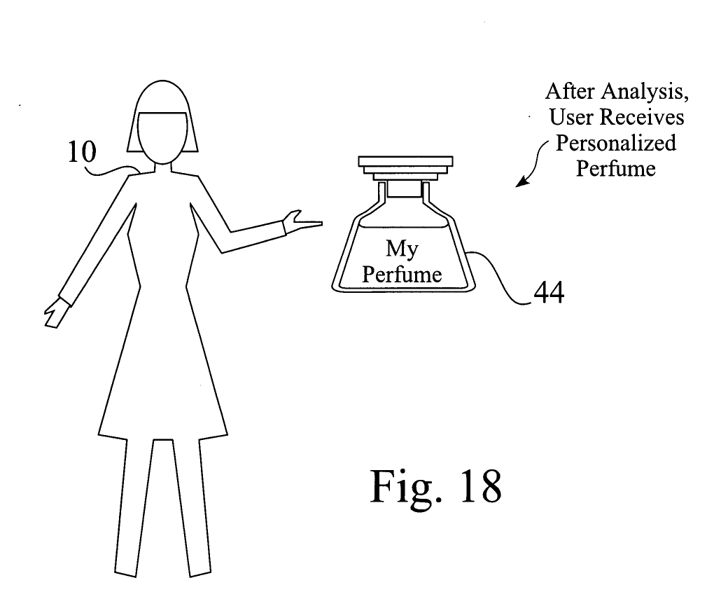

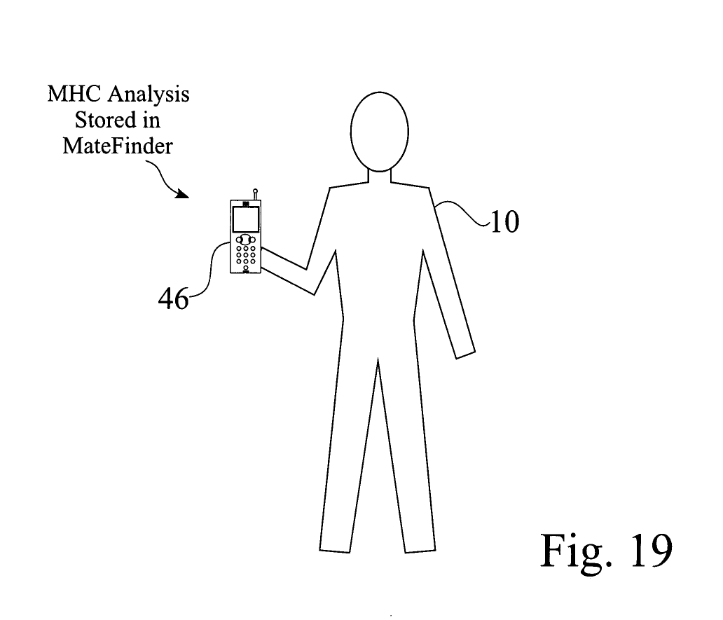

FIG. 19 shows a customer using a MateFinder™ device 46 which has been programmed with his or her genetic attributes, as determined in accordance with the present invention.

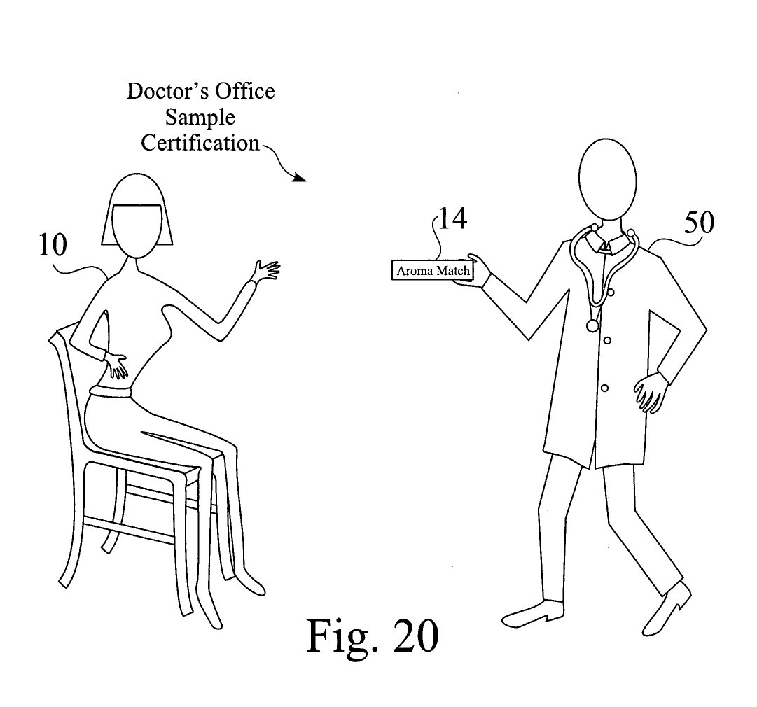

FIG. 20 shows a customer 10 visiting a doctor's office to provide a tissue sample, which is then certified by the doctor 50 before it is submitted to the laboratory. In this embodiment, the physician provides the test kit 14, and obtains the tissue sample. The physician then sends the tissue sample to a laboratory for analysis, and also certifies that the sample is from a particular person. In this example, the physician acts as a “notary” who insures the identity of the source of the sample. This implementation of the invention guards against the fraudulent submission of a tissue sample from a person who might attempt to supply a misleading identity.

II. Custom-Fabricated Perfumes

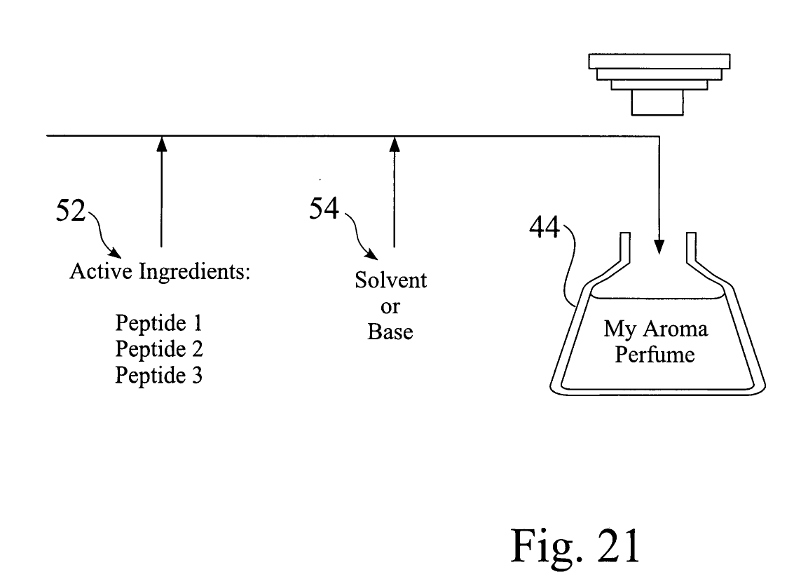

FIG. 21 depicts a method of manufacturing a customized perfume 44. General methods for manufacturing compositions for dispensing fragrances, aromas and perfumes are well known in the art. According to the Scented Products Education and Information Association of Canada, ingredients in a typical fragrance “recipe” generally include:
“extracts from plants and flowers (naturals), synthetic recreations (synthetic duplications of natural fragrance materials), synthetic innovations (variations of naturally-occurring materials which have unique olfactory properties).
In general, typical fragrance formulae contain 100-350 ingredients, with an average concentration of usually less than 1%.
In a perfume, ethyl alcohol (of the same grade and purity as in alcoholic beverages) composes 50-90% of the product, purified water may constitute 5-20% of the product, with the fragrance component accounting from 10-30% of the finished product. Also present are UV inhibitors (to prevent degradation in the bottle) and any additional colouring agents.”
   SPEIAC, 20 Britannia Road East, Suite 102, Mississauga, Ontario L4Z 3L5
   See: http://www.cctfa.ca/scented/fragmat.htm

In one embodiment of the present invention, appropriate combinations of biological agents such as peptides or other substances are added as active ingredients 52 to a base 54 to a mixture, together with and/or any other suitable solvents, stabilizers, agents, preservatives, dispersants, inhibitors or components. In one embodiment, the base is a solvent, such as alcohol or water. These biological agents are selected to match a genetic attribute possessed by a person.

In one implementation, the perfume or cologne 44 made in accordance with the invention contains substances which are complementary to the user's Major Histocompatibility Complex (MHC profile), which will be attractive to the same user. In the same implementation, that person may ask a spouse or mate to wear this perfume or cologne 44, which pleases the person for whom the customized perfume or cologne was made. The present invention includes both perfume or cologne intended to be used during interaction with another, either by applying to the other's body or clothing, or to part of one's one body or clothing, or to another object which the user can smell during the interaction.

The biological agents may be selected to promote the responsivity of the person using the mixture, or may be selected to promote the responsivity of another person using the mixture. The biological agents in the mixture may be used to broadcast or indicate sexual compatibility, interest, awareness or attraction. As an alternative, the biological agents may be selected to promote confidence, self-esteem or the interest or attraction of another. The invention may be used to promote relationships between members of the opposite sex, or between members of the same sex.

In this Specification and in the Claims that follow, the term “perfume” encompasses any composition of matter that conveys, carries or otherwise transmits a scent, an aroma, an odor or any other olfactory or sensory stimulant or signal. This composition includes, but is not limited to, a perfume, a cologne, a salve or balm, a paste, an aerosol or non-aerosol spray, a gas or mixture of gases, a powder or some other dry preparation, or a cosmetic. This composition may or may not be consciously sensible. The cosmetic may include skin cream, lipstick, lip balm, nail polish, hair spray, make-up, gel, liquid, powder, ointment, colorant, or some other preparation that be applied to the body. The mixture is generally intended to be applied to, dispensed on or worn on the skin or hair, but may be applied on or used in conjunction with an article of clothing or some other object, which may be impregnated with the active ingredients. In yet another embodiment, the perfume 44 may be encapsulated or contained in a pill or medication that is taken internally, and which is then secreted through the skin or which causes a biological reaction which produces or mimics an odor. In another embodiment, the composition may be incorporated in a soap, body wash or cleanser, which effectively washes away natural scents, and then covers the body with a new scent. The mixture may also be dispensed using a variety of devices, including, but not limited to air fresheners, aroma-dispensing devices, candles and incense.

This specialized perfume 44 contains a strong preparation of personal peptides, enabling the user to “broadcast” his or her “MHC” over a wide area, and increasing his or her chances of meeting a compatible partner. The perfume 44 provides a much more specific set of aromas than, for instance, not bathing for a long period, since odors thus derived have been bacterially modified and contain many non-MHC-related components.

The MHC is a cluster of genes that determines details of cellular surfaces and thus immune responses, and specifies certain peptides that appear in skin secretions and urine. These peptides are responsible for odors which uniquely identify individuals who are not identical twins. Detailed information concerning the MHC may be found in Leslie A. Knapp's publication entitled The ABCs of MHC, published in Evolutionary Anthropology 14:28-37 (2005) Wiley-InterScience. MyAroma™, MyPerfume™, MyEssence™ are Trade & Service Marks owned by the Assignee of the Present Patent Application.

III. Alternative Perfume & Matching Service Embodiments

Custom Perfume Including Standard Perfume Ingredients

In another embodiment of the invention, standard ingredients are used to compose a perfume. Individuals with similar MHC genes have similar preferences for standard perfume scents such as rose, vanilla, and musk. According to this implementation of the invention, one member of a couple sends his partner's MHC information for laboratory analysis, the manufacturer can design a perfume for him to give to her (or vice versa). More information concerning preferences for standard scents may be found in “Evidence for MHC-correlated Perfume Preferences in Humans” by Milinski, M. & Wedekind, C., Behavioral Ecology, 12, p. 140-149 (2001).

EssenceSniffer

In yet another embodiment, a user may receive several scent samples, based on common identified MHC-advertising scents. These samples are be placed on a single card (SnifferCard or EssenceCard), or on a series of cards sent in individual wrappers that preserve the scents and prevent cross-contamination. The customer would then rates these scents, sends her or his ratings to the laboratory, and then the MHC type to which she or he is attracted is determined. The customer is then matched based on this MHC type. A perfume including these characteristics made also be produced for her or his current partner to wear.

Service Provided to Parties Involved in Arranged Marriages

In many parts of the world, marriages are arranged by parents, elders, or other parties. These marriages are sometimes very unhappy, fail to produce children, or are dissolved completely. One source of failure is likely to be MHC mismatching, particularly in populations with some degree of inbreeding (leading to more MHC similarity than would otherwise occur). In another implementation of the invention, the parties involved in arranging marriages (e.g., parents or matchmakers) submit samples from marriage candidates to determine whether they are biochemically compatible, thus increasing the chances that the pair will be well-matched and will produce viable offspring.

Service Provided to Individuals with the ABCC11 Gene

New evidence suggests that 80-95% of East Asians have an allele of the ABCC11 gene which may inhibit axillary secretions, making scent discrimination more difficult. In this embodiment of the invention, the presence of this gene is determined. A customer is then alerted that their MHC advertising may be suppressed, and they may wish to use the stronger formulations of MHC-advertising perfume to attract reproductively compatible mates. More information concerning the ABCC11 Gene may be found in “The Dimorphism in Human Normal Cerumen” by Matsunaga, E., published in Annual Review of Human Genetics, 25, 273-286 (1962) and in “A SNP in the ABCC11 Gene is the Determinant of Human Earwax Type” by Yoshiura, K. et al., published in Nature Genetics, 29 January, doi: 10.1038/ng1733 (2006).

IV. A General Description of Sample Analysis, DNA Identification & Perfume Formulation

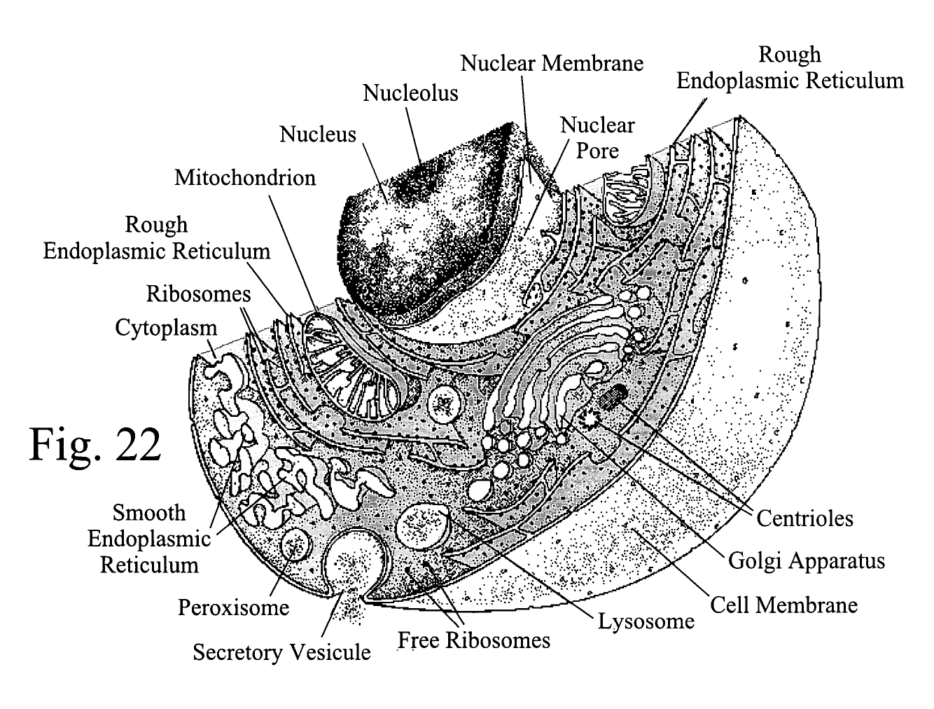

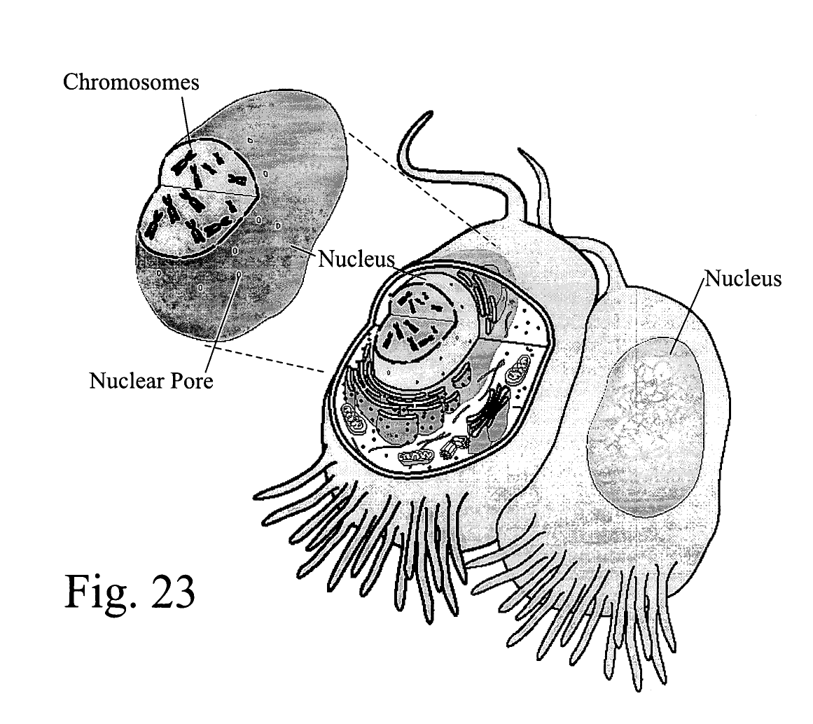

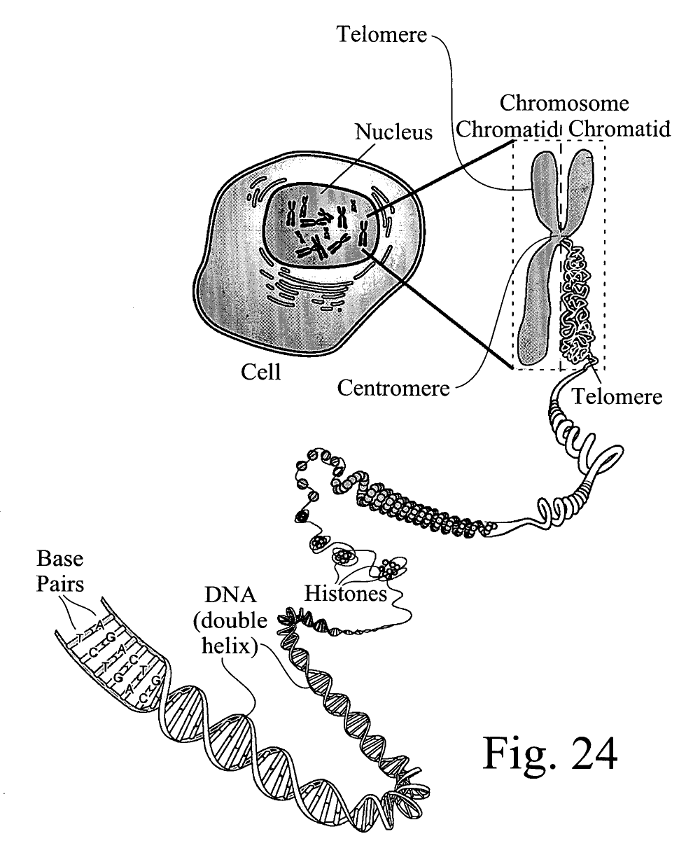

After a sample is received at the laboratory, it is processed to extract DNA. DNA, which is short for “deoxyribonucleic acid,” is the chemical inside the nucleus of a cell that carries the genetic instructions for making living organisms. A cell, which is shown in FIG. 22, is the basic unit of any living organism. It is a small, watery, compartment filled with chemicals and a complete copy of the organism's genome. Each cell contains a nucleus, which is portrayed in FIG. 23. The nucleus is the central cell structure that houses the chromosomes. Chromosomes are one of the threadlike “packages” of genes and other DNA in the nucleus of a cell. Chromosomes are shown enclosed within the nucleus, which is, in turn, enclosed in the center of the cell, as illustrated in FIG. 24.

Different kinds of organisms have different numbers of chromosomes. Humans have 23 pairs of chromosomes, 46 in all: 44 autosomes and two sex chromosomes. Each parent contributes one chromosome to each pair, so children get half of their chromosomes from their mothers and half from their fathers.

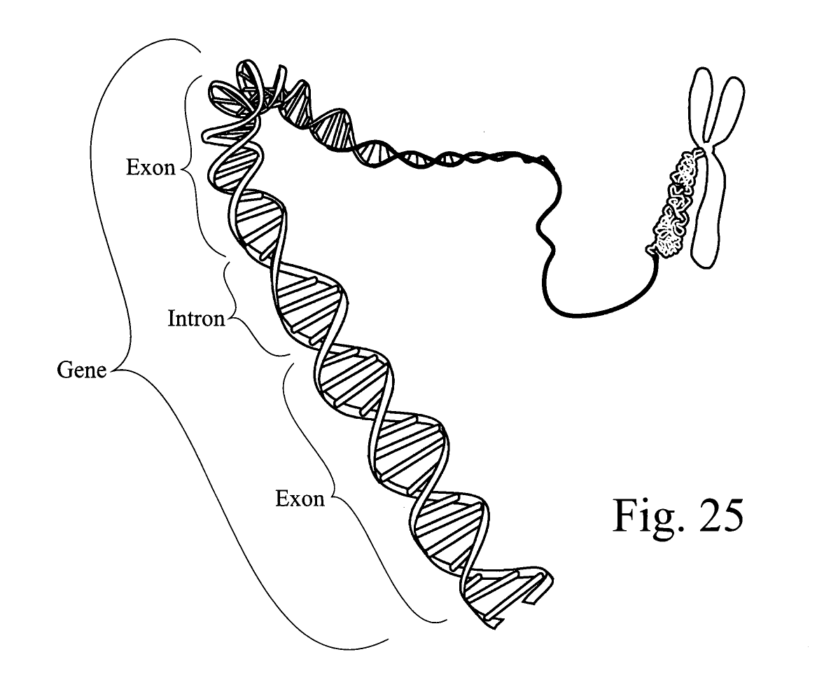

Part of the chromosome is called a gene, as depicted in FIG. 25. The gene is the functional and physical unit of heredity passed from parent to offspring. Genes are pieces of DNA, and most genes contain the information for making a specific protein. FIG. 25 shows how a length or strand of DNA forms a gene.

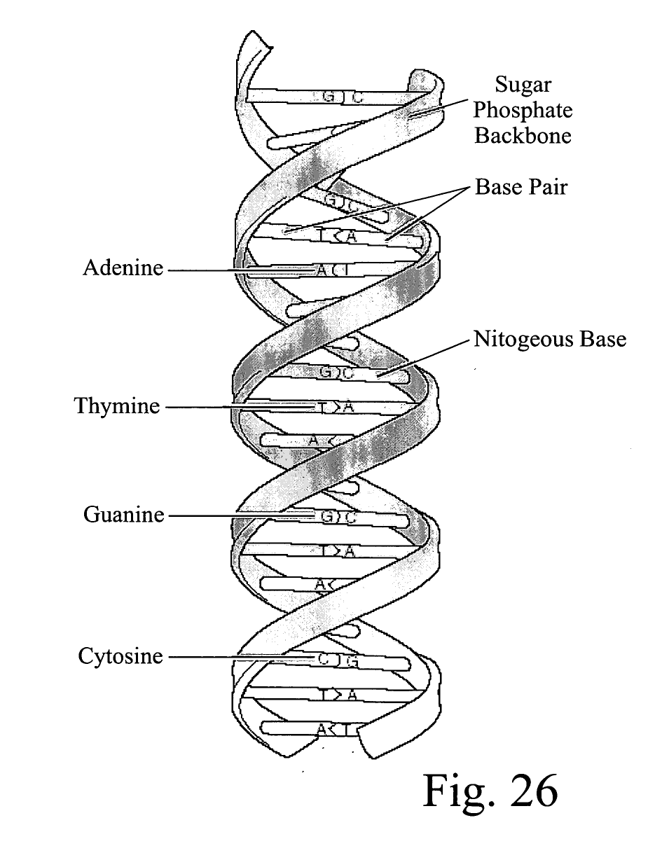

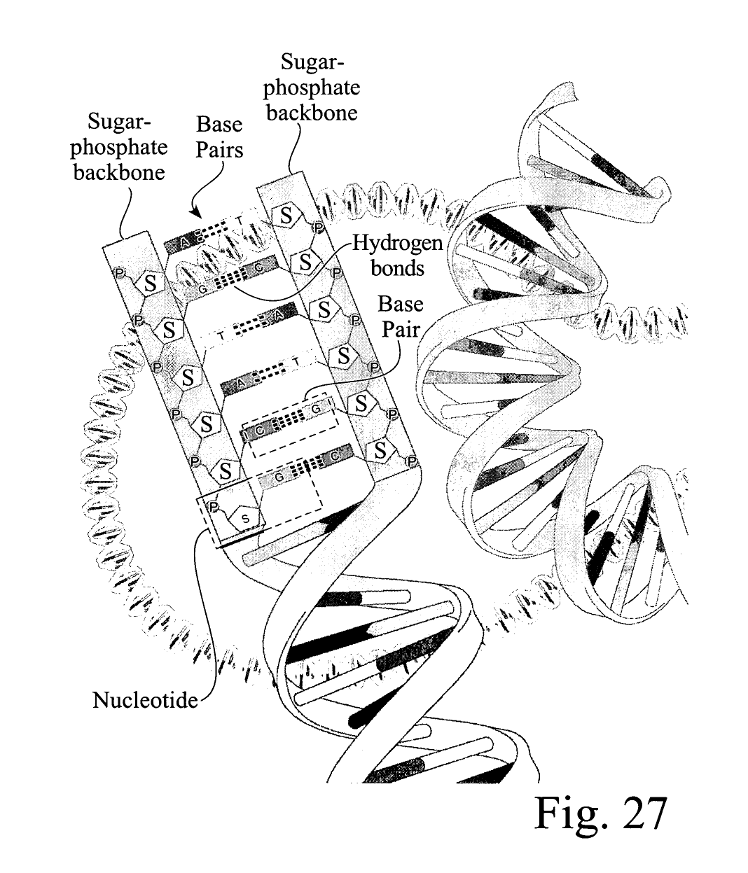

FIGS. 26 and 27 furnish views of a strand of DNA, which comprises a pair of helical ribbons attached by bases that resemble the rungs of a ladder. These bases are named adenine, thymine, guanine and cytosine. Sometime uracil is substituted for thymine. A section of one of the spiral sides of the DNA together with one of the bases comprises a nucleotide, which is shown in FIG. 27. Nucleotides are one of the structural components, or building blocks, of DNA and RNA. A nucleotide consists of a base (one of four chemicals: adenine, thymine, guanine, and cytosine) plus a molecule of sugar and one of phosphoric acid.

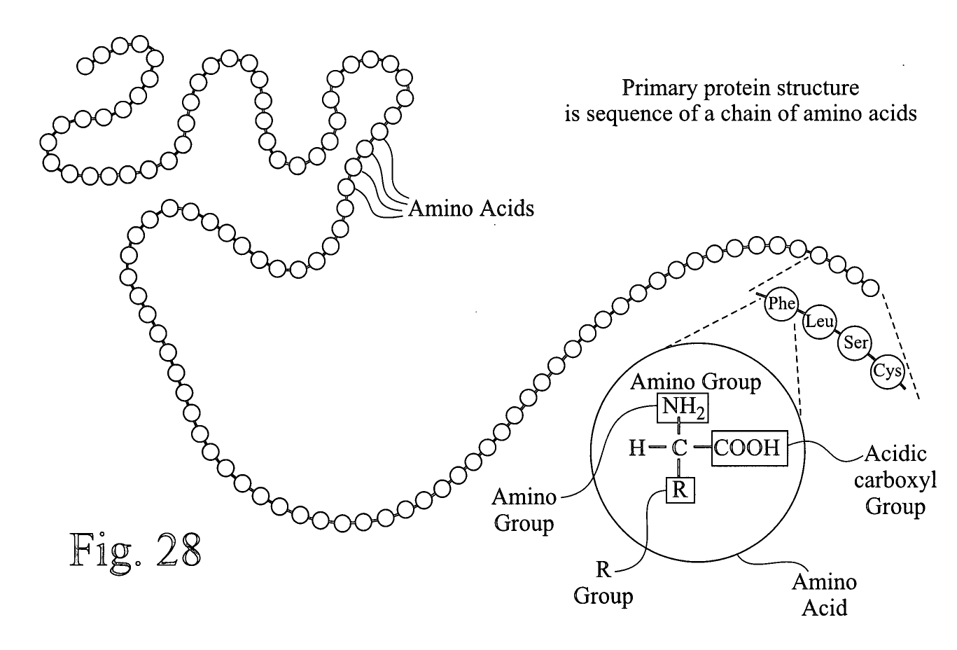

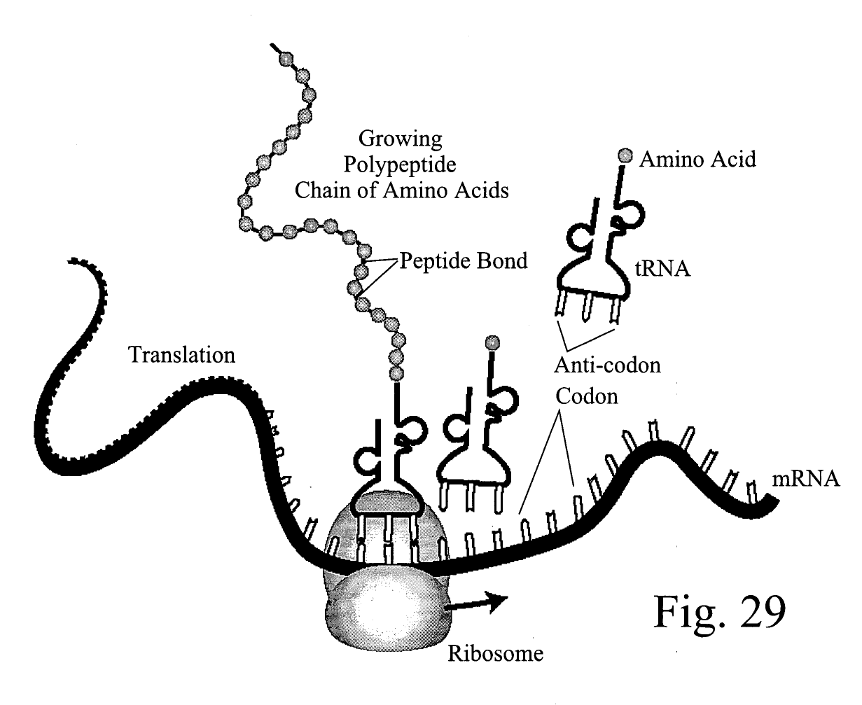

Another set of chemicals that are important building blocks in humans are amino acids. Amino acids are the “building blocks” of proteins, and are shown in FIG. 28. There are twenty different kinds of amino acids in the human body. When two or more amino acids are bonded together, they form a peptide, which is shown in FIG. 29.

An allele is one of the forms of a gene at a particular location or “locus” on a chromosome. Alleles are specific sequences of base pairs that can be present at a given locus. For example, at the HLA-A locus in a particular individual, alleles in the A*01 and A*02 groups may be found. The “*” in the allele group name indicates that it was determined by DNA typing, as opposed to serological methods.

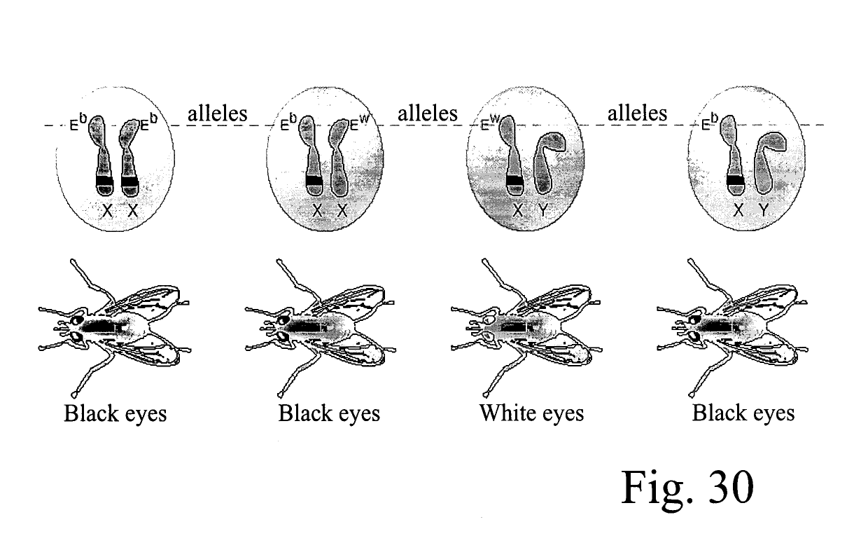

Different alleles produce variation in inherited characteristics such as hair color or blood type. In an individual, one form of the allele (the dominant one) may be expressed more than another form (the recessive one). A set of different alleles that reside on the chromosomes of a common fly are illustrated in FIG. 30. Genes may also be co-dominant, in which case both are expressed, as opposed to dominant/recessive genes, in which case only the dominant one is expressed.

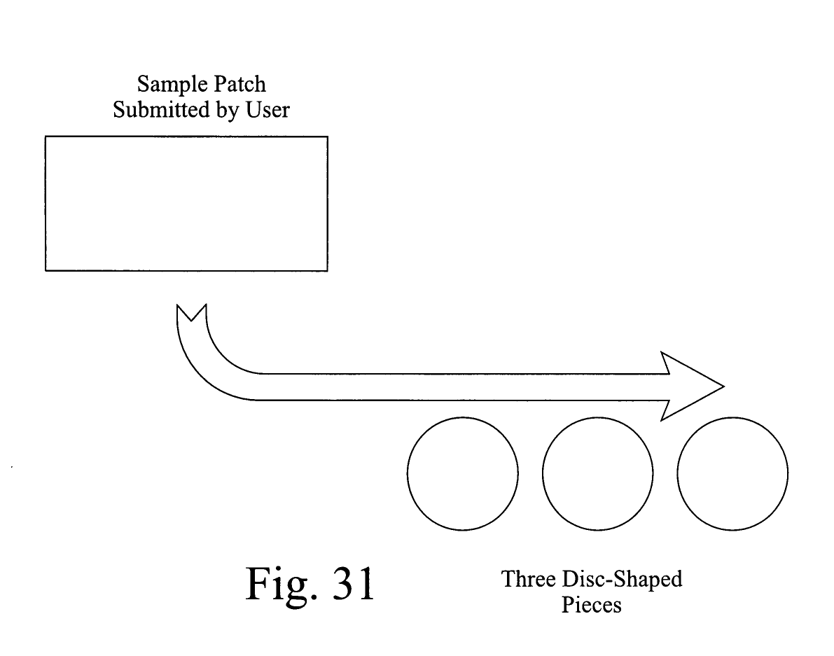

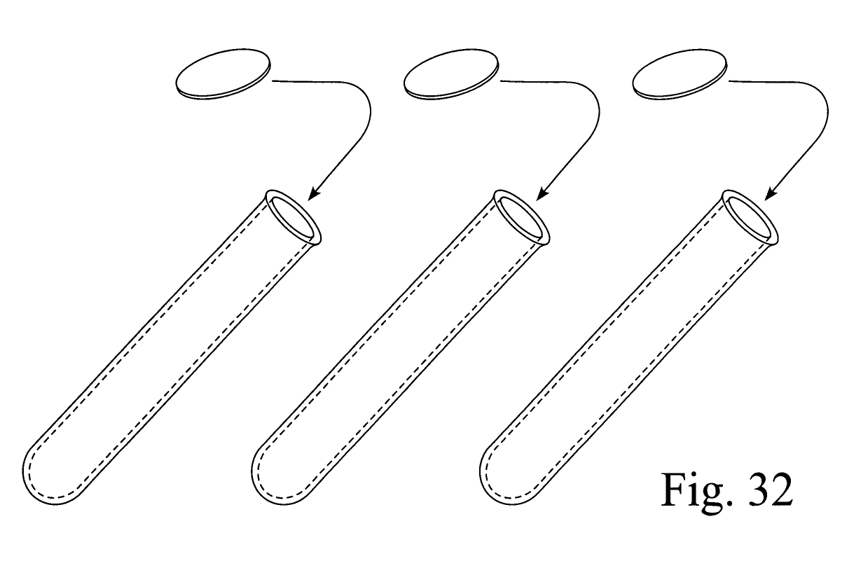

In accordance with the present invention, small amounts of DNA are obtained from the sample submitted to a laboratory by a user who has submitted a saliva sample or skin scraping. In one embodiment of the invention, personnel at the lab cut the sample using a punch to make three separate disc-shaped pieces, as shown in FIG. 31. These pieces are each placed in a different test tube as shown in FIG. 32. All the pieces are washed several times with chemicals that purify the sample on each piece. After washing, each piece is dried in its tube.

In an alternative embodiment, if a saliva sample is obtained from the customer, the saliva is poured directly into three separate test tubes, washed and then the DNA analysis is performed.

When DNA is analyzed, a laboratory technician looks at particular places or “loci,” (which are the positions in a chromosome in which specific genes are known to occur) to determine the particular allele (variation of the gene). Previous research has determined that every person has a characteristic sequence of genetic material (allele) that resides at each of his or her genetic loci.

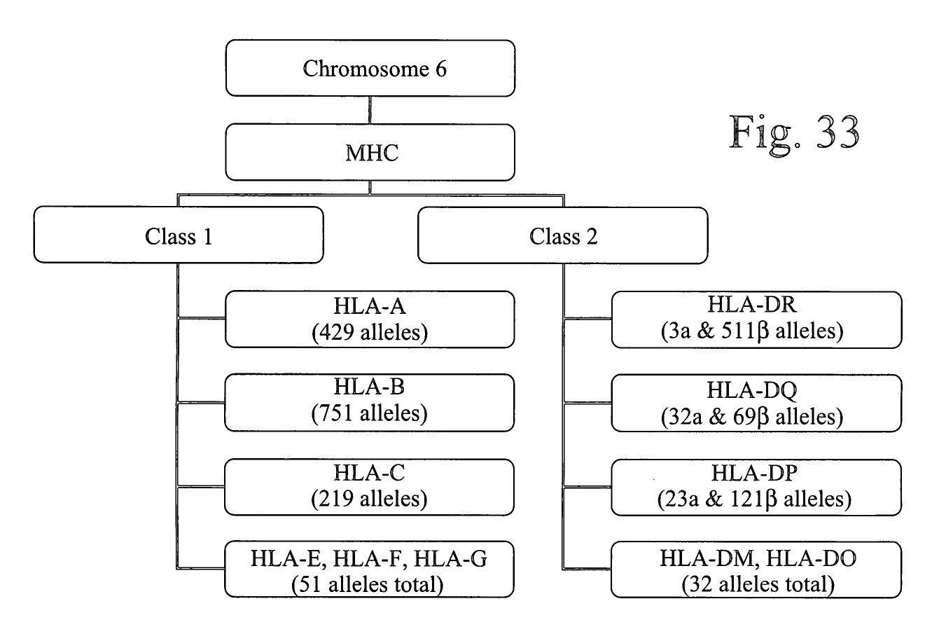

The laboratory technician basically examines particular sets of alleles that are found at a particular group of loci on a particular chromosome. Specifically, the technician “takes an inventory” of the genetic material in the MHC region on Chromosome 6. Parts of the MHC are broken down into smaller groups of genetic material, and are given names. The parts of the MHC that need to be inventoried are named “HLA-A,” “HLA-B” and “HLA-DRbeta1.” These parts of the MHC are correlated with particular regions of a particular chromosome. All these relationships are illustrated in FIG. 33.

The term “allele groups” are also known as “2-digit alleles” and “2 alleles.” “High resolution alleles” are also known as “4-digit alleles” and “4 alleles.”

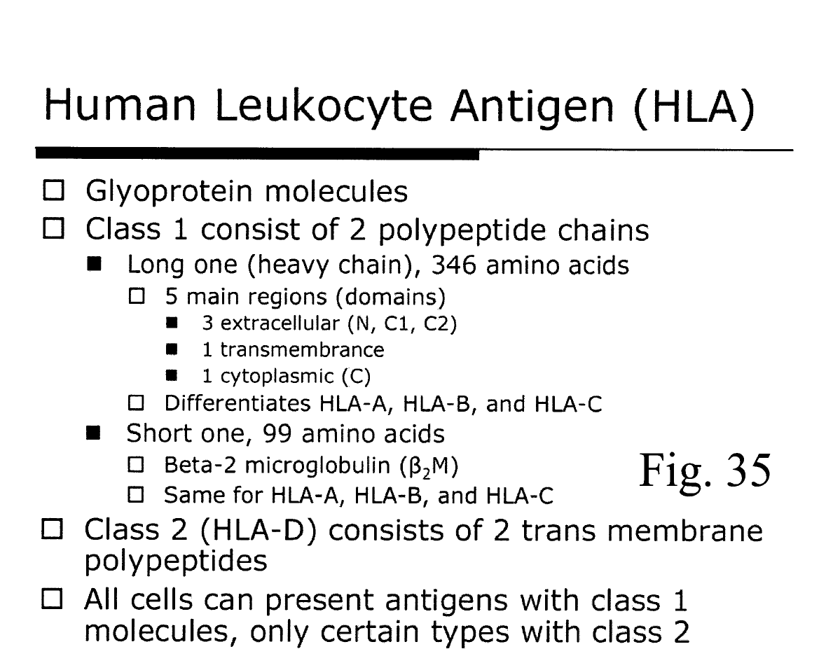

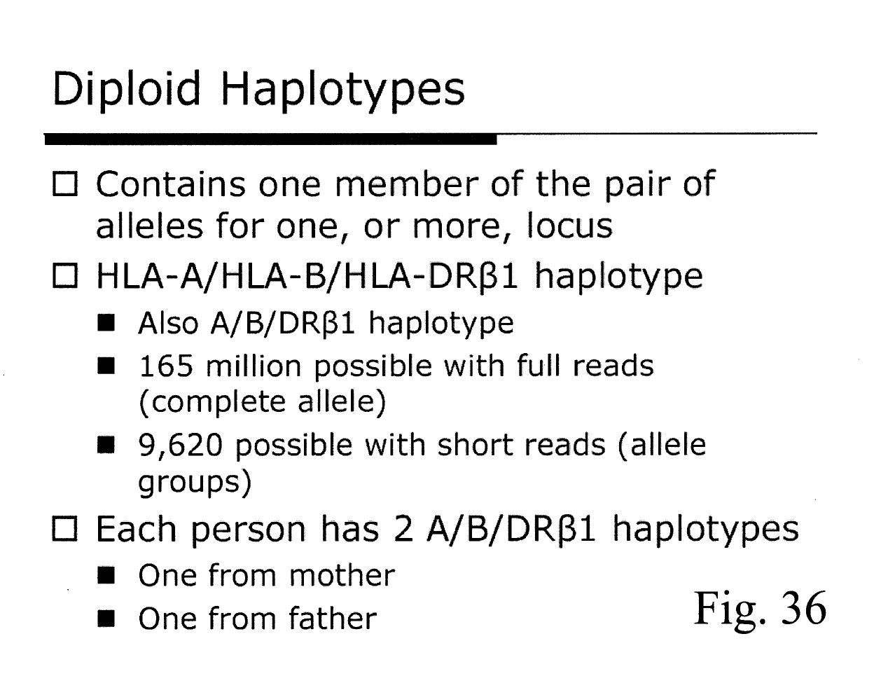

FIG. 35 describes a Human Leukocyte Antigen. FIG. 36 describes Diploid Haplotypes.

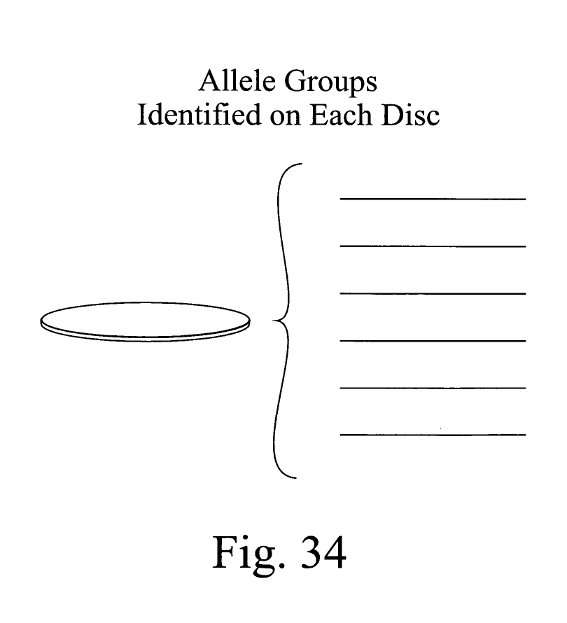

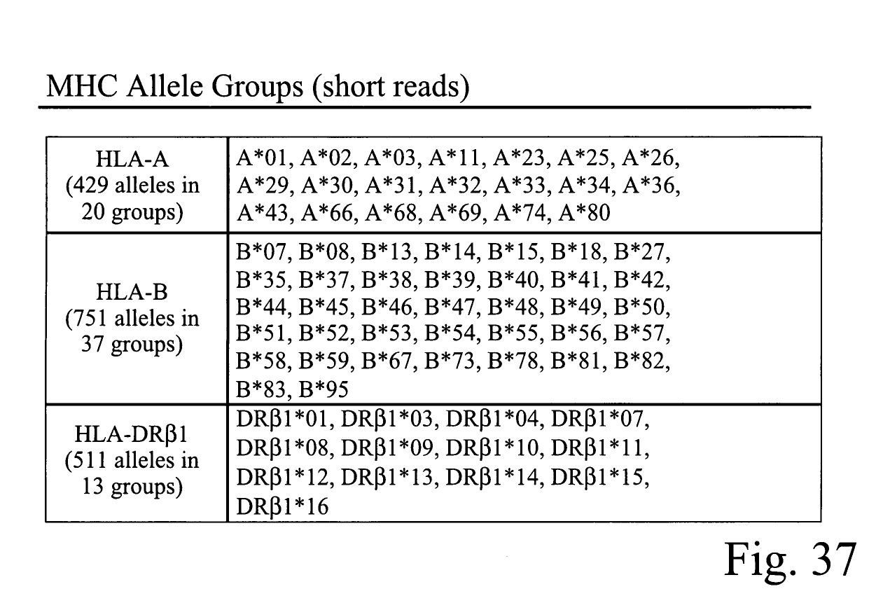

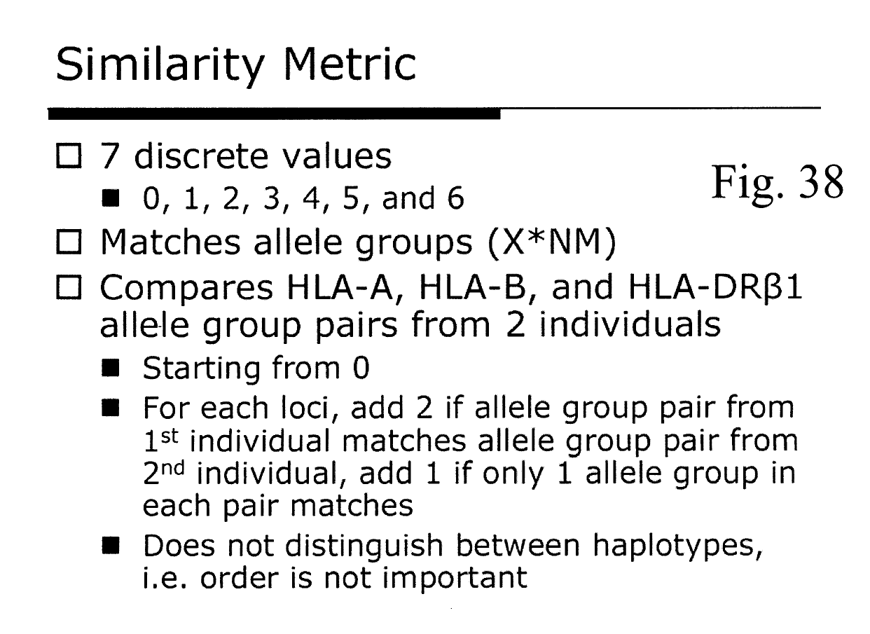

There are 21 HLA-A allele groups, 37 HLA-B allele groups, and 13 HLA-DRbeta1 allele groups. FIG. 34 is a schematic representation of the results of an analysis of one of the disc-shaped samples. The analysis provides a listing of allele groups. The various MHC Allele Groups, such as “A*01,” “A*02” and “A*03” are presented in FIG. 37. FIG. 38 describes a Similarity Metric.

The sequence-specific oligonucleotide probe (SSOP) method is used. The basis of this method is HLA locus-specific amplification by polymerase chain reaction (PCR), and the subsequent probing of the resulting product by SSOP. A battery of probes is required. The pattern of reaction to these probes distinguishes the HLA alleles.

For each sample, the laboratory uses PCR for HLA locus-specific amplification at HLA-A, HLA-B, and HLA-DRbeta1. Each of the three PCR amplifications results in a product. Each of the three products is then tested with a battery of probes. The HLA-A amplified product is tested with 12 probes at exon 2 and 16 probes at exon 3. The HLA-B amplified product is tested with 18 probes at exon 2 and 18 probes at exon 3. The HLA-DRbeta1 amplified product is tested with 25 probes at exon 2. These are sufficient numbers of probes so that the reaction patterns will distinguish the HLA allele groups (2-digit alleles), for example, A*02.

After all the genetic codes that are contained on a sample piece is identified, this information is entered into a database along with the personal information and match preferences of the customer who submitted the sample.

Previous scientific research has determined that a woman's sexual response is based on the correlation between the alleles in the woman's MHC, and in a man's MHC. Specifically, a woman and a man who have different MHC genetic patterns are more sexually compatible than a man and a woman who have similar MHC genetic codes.

So, when the lab technician takes an inventory of all the different allele groups (2-digit alleles) in a user's DNA sample, the technician is creating an identification or map of the person who submitted the sample. By comparing this identification or map with that of a different person, a technician can predict which other people will be attractive and sexually responsive to the customer, all based on the genetic code of each individual. In addition to the HLA-A, HLA-B and HLA-DRbeta1 loci specified above, genetic information from other loci on Chromosome 6 or any other chromosome may be used to enhance a match.

In alternative embodiment of the invention, genetic attributes are determined by analyzing serologically typed HLA antigens. While “allele groups” are determined by genetic testing, such as PCR-SSOP, HLA antigens are determined by serological, or blood reaction, testing. Serological typing provides approximately the same resolution as “2-digit alleles.” It cannot provide the higher resolution comparable to “4-digit alleles.”

More detailed information concerning this analysis may be found in “Methods in Molecular Biology, Vol. 210: MHC Protocols,” edited by S. H Powis and Robert W. Vaughan, Humana Press Inc., Totowa, N.J., 2003. (See Chapter 5, “PCR-Sequence-Specific Oligonucleotide Probe Typing for HLA-A, -B, and -DR, by Derik Middleton and F. Williams). Another useful publication is “Histocompatibility Testing,” edited by Jeffrey L. Bidwell and Cristina Navarrete, Imperial College Press, 2000. (See Chapter 6, “PCR-SSOP Typing” by D. Middleton.) These publications explain how to type the MHC loci of interest using a two-tier system. The first level of resolution determines the allele group (2-digit alleles), and the second level uses this knowledge to determine the allele subgroup (4-digit alleles).

HLA typing can be performed by the complement dependent lymphocytotoxicity reaction (serology). Live peripheral blood mononuclear cells are required for this assay (CD8+ T-cells and/or CD19+). B-cells are purified from whole blood, and incubated against a panel of antibodies with specificity against polymorphic epitopes expressed on HLA-A and -B proteins. In the presence of complement cells expressing HLA proteins which react with a particular antibody are lysed, allowing these damaged cells to uptake a stain which is detected by fluorescent microscopy. The pattern of negative and positive reactions is scored and interpreted to give an HLA serological type.

Additional information concerning this analysis may be found in “Histocompatibility Testing,” edited by Jeffrey L. Bidwell and Cristina Navarrete, Imperial College Press, 2000. (See Chapter 1, “HLA Typing by Alloantibodies and Monoclonal Antibodies” by G. M. Th. Schreuder; and Chapter 2, “Screening for HLA-Specific Antibodies” by C. Brown and C. Navarrete.) These publications explain how to type the MHC loci of interest using antibody reactions.

V. A Detailed Description of a Specific Embodiment of the Invention

In one particular embodiment of the invention, DNA samples are collected from a number of human individuals. A set of genetic attributes are determined for each of the individuals in this group. These genetic attributes may be entered and/or stored in a database. Based on the analysis of genetic attributes, a first and a second individual from the group are matched based on a compatible correlation of their genetic attributes. This matching promotes the sexual responsivity between the first and second individuals. A set of filtering rules may be employed to customize the matches by either restricting or promoting particular matches, based on criteria delineated by individuals in the group.

In addition, a perfume may be manufactured based on the analysis of the genetic attributes. The perfume may be used to promote the romantic self-confidence of the individual who wears the perfume, may be used to broadcast genetic attributes to another individual, or may be used promote the romantic interest of another human individual.

In one implementation of the invention, only a single attribute in the set of genetic attributes that are analyzed. In another embodiment, more than one attribute is analyzed.

The first and the second individuals may be humans, and may be members of the opposite sex, or may be members of the same sex. In an alternative embodiment of the invention, this method may be used in the veterinary field to enhance the selective breeding of livestock, pets or other animals.

In one specific embodiment of the invention, the set of genetic attributes are determined by analyzing a gene in the Major Histocompatibility Complex Region of Chromosome 6.

In a first case, a set of genetic attributes of the individuals in the group is determined by an analysis of alleles at the HLA-A locus. The alleles at the HLA-A locus may be measured to the resolution of an allele group, or to the resolution of an allele sub-group.

In a second case, a set of genetic attributes of the individuals in the group is determined by an analysis of alleles at the HLA-B locus. The alleles at the HLA-B locus may be measured to the resolution of an allele group, or to the resolution of an allele sub-group.

In a third case, a set of genetic attributes of the individuals in the group is determined by an analysis of alleles at the HLA-DRbeta1 locus. The alleles at the HLA-DRbeta1 locus may be measured to the resolution of an allele group, or to the resolution of an allele sub-group.

In one implementation, the set of genetic attributes is determined by an analysis of alleles at the HLA-A loci, the HLA-B loci and the HLA-DRbeta1 loci. The alleles at the HLA-A, HLA-B and HLA-DRbeta1 loci may be measured to the resolution of an allele group (2-digit allele), or to the resolution of an allele sub-group (4-digit allele).

The analysis is used to calculate a matching score between the first and second individuals. In one specific situation, this matching score is computed as a number of HLA-A alleles that the first individual has in common with the second individual; plus a number of HLA-B alleles that the first individual has in common with the second individual; plus a number of HLA-DRbeta1 alleles that the first individual has in common with the second individual.

In one case, the first and second individuals are matched based on a low matching score. In another case, the first and second individuals are matched based on a high matching score. The matching score ranges from zero, corresponding to no alleles in common, to six, corresponding to all alleles in common.

One specific method of computing a matching score employs the following steps:

initializing a score to zero;

then, for each locus, adding 2 if two alleles from said first individual match two alleles from said second individual at subgroup resolution;

adding 1.5 if one allele from said first individual matches one allele from said second individual at subgroup resolution and the other alleles match only at the group resolution;

adding 1 if one allele from the first person matches one allele from the second person at the subgroup resolution and the other alleles do not match;

adding 1 if the two alleles from the first person matches the two alleles from the second person at the group resolution only;

adding 0.5 if one allele from the first person matches one allele from the second person at the group resolution only and the other alleles do not match;

adding 0 otherwise; and

repeating for each of the three loci.

The genetic attributes which are analyzed may be related to one, or more, characteristics of physical appearance. In one specific instance, the genetic attributes may be related to eye color. In particular, the genetic attributes which are analyzed may be the two alleles at the EYCL1 locus on Chromosome 19, the two alleles at the EYCL2 locus located on Chromosome 15, and the two alleles at the EYCL3 locus on Chromosome 15.

In another specific instance, the genetic attributes are related to hair color. In particular, the genetic attributes of each individual are the two alleles at the HCL1 locus on Chromosome 19, the two alleles at the HCL2, locus on Chromosome 4, and the two alleles at the HCL3 locus on Chromosome 15.

In another specific instance, the genetic attributes are related to genetic diseases.

In one embodiment of the invention, the DNA sample is obtained using a self collected sample of biological material. In one specific implementation, the biological material is saliva. A sample of saliva may be obtained having a customer spit into a sample collection container. The container is then sealed, and is submitted to a laboratory by mail or by some other form of delivery.

As an alternative, saliva and/or checks cells are collected from the mouth using a swab. The saliva is transferred to a sample card by rubbing the swab on said card. The card is then sealed in an envelop with a desiccant, and then submitted to a laboratory.

VI. Security of DNA Samples

Business-to-Business Embodiment

In one embodiment of the invention, a subscriber to a dating service pays for and receives a Sample Test Kit from the dating service in the mail. The Sample Test Kit contains a collection kit and a mailing package that has a business-reply mailing label attached. One example of the Sample Test Kit is the Oragene™DNA Self-Collection Kit, which is manufactured by DNA Genotek. See http://biocompare.com.

The DNA sample is submitted to a laboratory, or to some other business entity, for analysis. The collection kit carries a bar code and no other identification. In the package there is also a card, bearing the same bar code, with spaces for the user's name, address, etc. The card comes with a business-reply envelope addressed to the dating service. The user collects the sample, fills out the card, and mails the sample and card separately.

When the laboratory receives the sample, which is identified only with the barcode, it performs an analysis. The results of the analysis are stored in a database, and does not provide the results to the dating service, the user, or anyone else.

When the dating service receives the card, it scans the barcode and stores its number with its user's personal information.

When it chooses, the dating service sends the laboratory the user's barcode number along with those of prospective matches. The laboratory compares the profiles of the various samples, stored in its database, and returns a report to the dating service with the degree of match of each requested sample to the user's sample. The dating service then recommends partners to the user based on the degree of match.

In this way, neither the laboratory nor the dating service can associate a particular genetic profile with a particular person, no matter how many times the data are compared.

Business-to-Consumer Embodiment

In this embodiment, the customer mails the sample and card as above, except that the card is mailed to a national audit firm, such as Ernst & Young. It includes a section in which the user states that he/she is using the information for mate selection purposes only, and that the sample really is his/hers. Users wishing to compare samples contact the audit firm, which in turn supplies the barcode numbers to the laboratory or to some other business entity, which informs the audit firm, which informs the informs the user of the results of the analysis, or alternatively informs the user of the degree of correspondence between the submitted sample and a second sample submitted by another user for analysis. It should be noted that such a second sample may already be in the laboratory's database or in a database held by another party.

In an alternative embodiment, the customer mails both the card and the sample to the laboratory or to some other business entity, but to separate post office boxes. Different people at the laboratory open the mail from each post office box, and the data remain sequestered forever.

### CONCLUSION

Although the present invention has been described in detail with reference to one or more preferred embodiments, persons possessing ordinary skill in the art to which this invention pertains will appreciate that various modifications and enhancements may be made without departing from the spirit and scope of the Claims that follow. The various alternatives for providing a Human Sample Matching System that have been disclosed above are intended to educate the reader about preferred embodiments of the invention, and are not intended to constrain the limits of the invention or the scope of Claims.

### LIST OF REFERENCE CHARACTERS

10 Woman
12 Retail store
14 AromaMatch™ Test Kit
16 Personal computer
18 Website: AromaMatch.com
20 Bottle of cleaning solution
22 Cotton ball
24 Sample patch
24C Central area of patch
24S Strips extending away from central area
25 Plaster
26 Antibiotic
27 Adhesive
28 Sealable plastic bag
30 Mailing envelope
32 Laboratory technician
34 Sample analyzer
36 Aroma in the air
38 Aroma-sampling kiosk
40 Shopping mall
42 Tissue sample from cheek swab
43 Spit cup
44 Custom-formulated perfume
46 MateFinder™ device
48 Genetic attributes stored in MateFinder
50 Medical doctor or independent laboratory technician
52 Active ingredient
54 Base

What is claimed is:

1. A method comprising the steps of:
collecting a DNA sample from each of a plurality of human individuals;
said plurality of human individuals including a first and a second human individual;
determining a set of genetic attributes for each of said human individuals;
selecting said first individual and said second individual based on a compatible correlation of each of their own said set of genetic attributes to promote sexual responsivity between said first and said second individual humans; and
producing a perfume for one of said human individuals based on one of said set of genetic attributes to promote romantic self-confidence.

2. A method as recited in claim 1, in which said set of genetic attributes includes a single attribute.

3. A method as recited in claim 1, in which said set of genetic attributes includes a plurality of attributes.

4. A method as recited in claim 1, in which said first individual and said second individual are members of the opposite sex.

5. A method as recited in claim 1, in which said first individual and said second individual are members of the same sex.

6. A method as recited in claim 1, in which said set of genetic attributes are determined by analyzing a gene in the Major Histocompatibility Complex Region of Chromosome 6.

7. A method as recited in claim 1, in which said set of genetic attributes of each of said plurality of individuals is determined by an analysis of alleles at an HLA-A locus.

8. A method as recited in claim 7, in which said HLA-A loci are measured to the resolution of an allele group.

9. A method as recited in claim 7, in which said HLA-A loci are measured to the resolution of an allele sub-group.

10. A method as recited in claim 1, in which said set of genetic attributes of each of said plurality of individuals is determined by an analysis of alleles at an HLA-B locus.

11. A method as recited in claim 9, in which said HLA-B loci are measured to the resolution of an allele group.

12. A method as recited in claim 7, in which said HLA-B loci are measured to the resolution of an allele sub-group.

13. A method as recited in claim 1, in which said set of genetic attributes of each of said plurality of individuals is determined by an analysis of alleles at an HLA-DRbeta1 locus.

14. A method as recited in claim 11, in which said HLA-DRbeta1 loci are measured to the resolution of an allele group.

15. A method as recited in claim 7, in which said HLA-DRbeta1 loci are measured to the resolution of an allele sub-group.

16. A method as recited in claim 1, in which said set of genetic attributes of each of said plurality of individuals is determined by an analysis of alleles at an HLA-A locus, an HLA-B locus and an HLA-DRbeta1 locus.

17. A method as recited in claim 13, in which said HLA-A, HLA-B and HLA-DRbeta1 loci are measured to the resolution of an allele group.

18. A method as recited in claim 13, in which said HLA-A, HLA-B and HLA-DRbeta1 loci are measured to the resolution of an allele sub-group.

19. A method as recited in claim 16, further comprising the step of:
calculating a matching score between said first and said second individual based on said genetic attributes of said first and said second individual.

20. A method as recited in claim 19, in which said matching score is computed as
a number of HLA-A alleles that said first individual has in common with said second individual;
plus a number of HLA-B alleles that said first individual has in common with said second individual;
plus a number of HLA-DRbeta1 alleles that said first individual has in common with said second individual.

21. A method as recited in claim 20, in which said first and said second individuals are matched based on a low matching score of two or less.

22. A method as recited in claim 20, in which said first and said second individuals are matched based on a high matching score of four or more.

23. A method as recited in claim 1, in which said set of genetic attributes are related at least one characteristic of physical appearance.

24. A method as recited in claim 23, in which said characteristic of physical appearance is eye color.

25. A method as recited in claim 1, in which said genetic attributes are determined by analyzing two alleles at the EYCL1 locus on Chromosome 19, two alleles at the EYCL2 locus located on Chromosome 15, and two alleles at the EYCL3 locus on Chromosome 15.

26. A method as recited in claim 23, in which said characteristic of physical appearance is hair color.

27. A method as recited in claim 26, in which said set of genetic attributes are determined by analyzing two alleles at the HCL1 locus on Chromosome 19, two alleles at the HCL2, locus on Chromosome 4, and two alleles at the HCL3 locus on Chromosome 15.

28. A method as recited in claim 1, in which said set of genetic attributes are related to genetic diseases.

29. A method as recited in claim 1, in which said set of genetic attributes are determined using a self-collected sample of biological material.

30. A method as recited in claim 29 in which said biological material is saliva.

31. A method as recited in claim 30, further comprising the steps of:
spitting into a sample collection container;
sealing said sample collection container; and
submitting said sample container for analysis.

32. A method as recited in claim 30, further comprising the steps of:
collecting saliva from the mouth using a swab;
transferring said saliva to a sample card by rubbing said swab on said sample card;
sealing said card in an envelope with a desiccant; and
submitting said envelope for analysis.

33. A method as recited in claim 1, in which said set of genetic attributes of said plurality of human individuals is entered in a database.

34. A method as recited in claim 1, in which said set of genetic attributes of said plurality of human individuals is stored in a database.

35. A method as recited in claim 1, in which said set of genetic attributes of said first person is entered into a database.

36. A method as recited in claim 1, in which said set of genetic attributes of said first person are stored in a database.

37. A method as recited in claim 1, in which said set of genetic attributes of said second person is entered into a database.

38. A method as recited in claim 1, in which said set of genetic attributes of said second person are stored in a database.

39. A method as recited in claim 1, in which the comparison of said set of genetic attributes is used to prevent certain human individuals from among said plurality of human individuals from being matched with said first individual.

40. A method as recited in claim 1, in which the comparison of said set of genetic attributes is used to allow certain human individuals from among said plurality of human individuals to be matched with said first individual.

41. A method as recited in claim 1, further comprising the step of:
using a standard ingredient to compose said perfume.

42. A method as recited in claim 1, further comprising the steps of:
providing said first individual with a scent sample; said scent sample being based on common identified MHC-advertising scents;
obtaining a rating of said scent sample from said first individual; and
using said rating to determined which MHC type said first individual has an attraction; and
matching said first individual to said second individual based on a particular MHC type indicated by said rating.

43. A method as recited in claim 1, further comprising the step of:
matching said first individual and said second individual who are involved in an arranged marriage.

44. A method as recited in claim 1, further comprising the step of:
determining the presence of an ABCC11 Gene in said set of genetic attributes;
alerting said first individual to the presence of said ABCC11 Gene; and
providing a relatively strong formulation of said perfume to attract a reproductively compatible mate.

45. A method as recited in claim 1, further comprising the step of:
analyzing a serologically typed HLA antigen.

46. A method comprising the steps of:
collecting a DNA sample from each of a plurality of human individuals;
said plurality of human individuals including a first and a second human individual;
determining a set of genetic attributes for each of said human individuals;
selecting said first individual and said second individual based on a compatible correlation of each of their own said set of genetic attributes to promote sexual responsivity between said first and said second individual humans; and
producing a perfume for one of said human individuals based on one of said set of genetic attributes to promote the romantic interest of another human individual.

47. A method as recited in claim 46, in which said set of genetic attributes includes a single attribute.

48. A method as recited in claim 46, in which said set of genetic attributes includes a plurality of attributes.

49. A method as recited in claim 46, in which said first individual and said second individual are members of the opposite sex.

50. A method as recited in claim 46, in which said first individual and said second individual are members of the same sex.

51. A method as recited in claim 46, in which said set of genetic attributes are determined by analyzing a gene in the Major Histocompatibility Complex Region of Chromosome 6.

52. A method as recited in claim 46, in which said set of genetic attributes of each of said plurality of individuals is determined by an analysis of alleles at an HLA-A locus.

53. A method as recited in claim 52, in which said HLA-A loci are measured to the resolution of an allele group.

54. A method as recited in claim 52, in which said HLA-A loci are measured to the resolution of an allele sub-group.

55. A method as recited in claim 46, in which said set of genetic attributes of each of said plurality of individuals is determined by an analysis of alleles at an HLA-B locus.

56. A method as recited in claim 55, in which said HLA-B loci are measured to the resolution of an allele group.

57. A method as recited in claim 55, in which said HLA-B loci are measured to the resolution of an allele sub-group.

58. A method as recited in claim 46, in which said set of genetic attributes of each of said plurality of individuals is determined by an analysis of alleles at an HLA-DRbeta1 locus.

59. A method as recited in claim 58, in which said HLA-DRbeta1 loci are measured to the resolution of an allele group.

60. A method as recited in claim 58, in which said HLA-DRbeta1 loci are measured to the resolution of an allele sub-group.

61. A method as recited in claim 46, in which said set of genetic attributes of each of said plurality of individuals is determined by an analysis of alleles at an HLA-A locus, an HLA-B locus and an HLA-DRbeta1 locus.

62. A method as recited in claim 61, in which said HLA-A, HLA-B and HLA-DRbeta1 loci are measured to the resolution of an allele group.

63. A method as recited in claim 61, in which said HLA-A, HLA-B and HLA-DRbeta1 loci are measured to the resolution of an allele sub-group.

64. A method as recited in claim 1, further comprising the step of:
calculating a matching score between said first and said second individual based on said genetic attributes of said first and said second individual.

65. A method as recited in claim 64, in which said matching score is computed as
a number of HLA-A alleles that said first individual has in common with said second individual;
plus a number of HLA-B alleles that said first individual has in common with said second individual;
plus a number of HLA-DRbeta1 alleles that said first individual has in common with said second individual.

66. A method as recited in claim 65, in which said first and said second individuals are matched based on a low matching score of two or less.

67. A method as recited in claim 65, in which said first and said second individuals are matched based on a high matching score of four or more.

68. A method as recited in claim 46, in which said set of genetic attributes are related at least one characteristic of physical appearance.

69. A method as recited in claim 68, in which said characteristic of physical appearance is eye color.

70. A method as recited in claim 46, in which said genetic attributes are determined by analyzing two alleles at the EYCL1 locus on Chromosome 19, two alleles at the EYCL2 locus located on Chromosome 15, and two alleles at the EYCL3 locus on Chromosome 15.

71. A method as recited in claim 68, in which said characteristic of physical appearance is hair color.

72. A method as recited in claim 46, in which said set of genetic attributes are determined by analyzing two alleles at the HCL1 locus on Chromosome 19, two alleles at the HCL2, locus on Chromosome 4, and two alleles at the HCL3 locus on Chromosome 15.

73. A method as recited in claim 46, in which said set of genetic attributes are related to genetic diseases.

74. A method as recited in claim 46, in which said set of genetic attributes are determined using a self collected sample of biological material.

75. A method as recited in claim 74 in which said biological material is saliva.

76. A method as recited in claim 75, further comprising the steps of:
spitting a portion of saliva into a sample collection container;
sealing said sample collection container; and
submitting said sample container for analysis.

77. A method as recited in claim 75, further comprising the steps of:
collecting saliva from the mouth using a swab;
transferring said saliva to a sample card by rubbing said swab on said sample card;
sealing said card in an envelope with a desiccant; and
submitting said envelope for analysis.

78. A method as recited in claim 46, in which said set of genetic attributes of said plurality of human individuals is entered in a database.

79. A method as recited in claim 46, in which said set of genetic attributes of said plurality of human individuals is stored in a database.

80. A method as recited in claim 46, in which said set of genetic attributes of said first person is entered into a database.

81. A method as recited in claim 46, in which said set of genetic attributes of said first person are stored in a database.

82. A method as recited in claim 46, in which said set of genetic attributes of said second person is entered into a database.

83. A method as recited in claim 46, in which said set of genetic attributes of said second person are stored in a database.

84. A method as recited in claim 46, in which the comparison of said set of genetic attributes is used to prevent certain human individuals from among said plurality of human individuals from being matched with said first individual.

85. A method as recited in claim 46, in which the comparison of said set of genetic attributes is used to allow certain human individuals from among said plurality of human individuals to be matched with said first individual.

86. A method as recited in claim 46, further comprising the step of:
using a standard perfume ingredient to compose said perfume.

87. A method as recited in claim 46, further comprising the steps of:
providing said first individual with a scent sample; said scent sample being based on common identified MHC-advertising scents;
obtaining a rating of said scent sample from said first individual; and
using said rating to determined which MHC type said first individual has an attraction; and
matching said first individual to said second individual based on a particular MHC type indicated by said rating.

88. A method as recited in claim 46, further comprising the step of:
matching said first individual and said second individual who are involved in an arranged marriage.

89. A method as recited in claim 46, further comprising the step of:
determining the presence of an ABCC11 Gene in said set of genetic attributes;
alerting said first individual to the presence of said ABCC11 Gene; and
providing a relatively strong formulation of said perfume to attract a reproductively compatible mate.

90. A method as recited in claim 46, further comprising the step of:
analyzing a serologically typed HLA antigen.

91. A method as recited in claim 1, further comprising the step of:
broadcasting said first individual's own said set of genetic attributes.

92. A method as recited in claim 91, in which said first individual's own said set of genetic attributes are broadcast to attract a compatible mate.

93. A method as recited in claim 46, further comprising the step of:
broadcasting said first individual's own said set of genetic attributes.

94. A method as recited in claim 93, in which said first individual's own said set of genetic attributes are broadcast to attract a compatible mate.

95. A method as recited in claim 1, in which said perfume is incorporated in a soap.

96. A method as recited in claim 1, in which said perfume is incorporated in a body wash.

97. A method as recited in claim 1, in which said perfume is incorporated in a cleanser.

98. A method as recited in claim 46, in which said perfume is incorporated in a soap.

99. A method as recited in claim 46, in which said perfume is incorporated in a body wash.

100. A method as recited in claim 46, in which said perfume is incorporated in a cleanser.
